SEQUENCE LISTING (13 sequences)

SEQ ID NO: 1 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 2 - DNA, 4001 bp, Homo sapiens
aggcatcagcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctctaaaaaaaaaaaaaaaaa

SEQ ID NO: 3 - DNA, 20 bp, Artificial Sequence
  Description of Artificial Sequence Synthetic primer
cccaaaggaattccgagaaa

SEQ ID NO: 4 - DNA, 18 bp, Artificial Sequence
  Description of Artificial Sequence Synthetic primer
tcctttttggtgctgatg

SEQ ID NO: 5 - protein, 934 aa, Homo sapiens [sequence omitted]

SEQ ID NO: 6 - protein, 323 aa, Homo sapiens
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQKVRALGKNPRSWGKERAPNKHNLRPQSFKVKKEPKTRHSGFRL

SEQ ID NO: 7 - protein, 297 aa, Homo sapiens
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQKARRLFDVLHEPFLKVP

SEQ ID NO: 8 - DNA, 3145 bp, Homo sapiens
ggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtgg

SEQ ID NO: 9 - DNA, 1291 bp, Homo sapiens
gagccgaaggtggaggtcaaaggggcgtggcgttacagagcctctagcgctgggtgttggggacctgacgctatggagctctcggagttttgtgggggacggctgtgagtggggggttcctgctgcgggatgagaacgtagacgccagtggctcactcgctcctggcaccttccctttcaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccgggtgactgaatgtctgaccaccgtgaagtcagtcaacaaaacggacagtcagaccctcctgaccacatttggatctctggaacagctcatcgccgcatcaagagaagatctggccttatgcccaggcctgggccctcagaaagtaagagctctgggaaagaacccaaggagttgggggaaggagagagccccaaataaacacaacctgagaccccaaagttttaaggtgaaaaaagaaccaaagaccagacacagtggcttccgcctgtaatcccaacattttgggaggccaaggcgggaggactgcttgaggccagaagttggagaccagcctgggcaagtggacacctcatttttactaaaaataaaaaaaactagctgggcaaaaaaaaaaaaaaaaaa

SEQ ID NO: 10 - DNA, 3400 bp, Homo sapiens
ccggaagtgctgcgagccctgggccacgctggccgtgctggcagtgggccgcctcgatccctctgcagtctttcccttgaggctccaagaccagcaggtgaggcctcgcggcgctgaaaccgtgaggcccggaccacaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccgggtgactgaatgtctgaccaccgtgaagtcagtcaacaaaacggacagtcagaccctcctgaccacatttggatctctggaacagctcatcgccgcatcaagagaagatctggccttatgcccaggcctgggccctcagaaagcccggaggctgtttgatgtcctgcacgagcccttcttgaaagtaccctgatgaccccagctgccaaggaaacccccagtgtaataataaatcgtcctcccaggccaggctcctgctggctgcgctggtgcagtctctggggagggattctgggggtgtcaccttctggtggcccaggtgggcaccttcagctttctttagttcctcagtttcccgggggcagactacacaggctgctgctgctgctgcttccgcttcttgtcccggcctgtgggagcctcctccccagactctgaattcagtggcggccctggcatctcctcttggggcactgtctctggcatccggctttcctgactctgcttcttcctcttcttggtggatcccggagttgccctggcttcaggctgtccctcccctggcagttcaggctctagtggctgaattggctcagtcactgtgtgacctctctctttcttcttcttcttcttcttggtggatgtgggagctgcctgaggctcaaggtcatccggcagctcaggccccaccacctctgtctctggctccactgtggcatcttgctgtttttctttcttcgtcttctttttgggagctgccagagctgcctgggcctgaggcttcgctccttctggctgttgaggcgccatggtcccccctggggactccagaggcttcatctccggctccactggctccatcgcctccgtccctggctccatcattgccatctgtcccttttcttttttcctcttcttcgtagggggcagagggatggcttcctccagtggctccaccttcacctgtggctgagactcaactgtcaccccctcctctggctccatcccttccgtccccttttgcctctttctctttttggtcggggacaggactgtgtcttctagaggctcagtgttaatctgttcctgcttcactgtcttgtcttctggctcgaaggtttctttccctttgggcttcttcctcttcttggtggtggacgggaacagcactcccagaggctccagtgtctccactgtgggctctgtccccacaggccctgctgcctctggttctttcagctgctgattttttttcttcttcttcttccgcacatccatttctggcgaccccaaagccatgtccacctccagggccccgtgcccattcactgcctcctgagtgactggggcctctgtcacctgcatctcctttttcttcttccctgaggtgagcaggttgggggccaaggctgacctaggccctgtgactggtgggttgcccccaaaggcacagaaccgaggcctcaggccaggagggatctgtggtgggggacttgctgggatgggctgcagagggctccctgacagggattgctggggaccctcaaggatccttagggtgccctggggggctgaggcacaggtgagtccacctcctgcctccgttgagggggccagcagggtcgcttctccagcttggggacagctgctgaggactcgatagcggtgccgcttgcctgccaatttgcccttgacgatctgggagccagagagaggcacatgccgcccattgaagctacagagagaaacagggagggcagaggcttaagtggaacaggagagggaaggttttttgattttttttttgtttttttttgagagagtcttgctctgttgcctaggctggagtgcagtggcatgatctcggctcactgcaatgtccacctcctgggttcaagcgattctcctgcctcagcctctcaagtagctgggattacaggcacctgccaccacgcccagccaatttttgtatttttagtagagacaatttcactatgttggccaggctggtcttgaactcctgacctcaagtgatctgctcgcctcggcctcccaaaggatgggattacaggcaccagccactgcgcctggctggcctctggtttttaataaaacatgactagagtgactccatcttaaagtgagtagctaggcacttacaaggttcatgcttatggcctgaaaataaccacatcccaggctgaccaccaattataattacagaatatttatggccatacagaacatgttccaccaagcctgcagaatgtccaaatgtcctaagaatgcagcccccattacttaaatataacataaatgagcaagcttaggttgcaggattaatggtcgtggataacaccaatagcccctacctttagtgagcttatctgcacactccaagtttaactatagttccttatagtttcttataagtagaaatactaacaaagggctgtgggtttctccccctgctttctgaggacactctactctgtaaaggagtagtttccaataaacttgtttctttcactgtgcaaaaaaaaaaaaaaaaaa

SEQ ID NO: 11 - DNA, 21 bp, Artificial Sequence
  Description of Artificial Sequence Synthetic primer
gggaatttggcgacgtaattc

SEQ ID NO: 12 - DNA, 18 bp, Artificial Sequence
  Description of Artificial Sequence Synthetic primer
gcggaggctgaggaacag

SEQ ID NO: 13 - DNA, 25 bp, Artificial Sequence
  Description of Artificial Sequence Synthetic probe
cacaggtgctctggcccagcacata

(19) United States
(12) Patent Application Publication
(10) Pub. No.: US 2012/0277110 A1
(43) Pub. Date: Nov. 1, 2012

(54) PARP AND ADJUVANT CISPLATIN-BASED CHEMOTHERAPY IN NON-SMALL-CELL LUNG CANCER

(76) Inventors: Fabrice Andre, Sceaux (FR); Ken André Olaussen, Paris (FR); Jean-Charles Soria, Igny (FR)

(21) Appl. No.: 13/386,474

(22) PCT Filed: Jul. 26, 2010
(86) PCT No.: PCT/EP10/60819

§ 371 Date: Jan. 23, 2012

Related U.S. Application Data
(60) Provisional application No. 61/228,341, filed on Jul. 24, 2009.

(51) Int. Cl. C40B 30/04; C40B 40/10; G01N 33/573; C40B 40/06; C12Q 1/48; C12Q 1/68

(52) U.S. Cl. 506/9; 435/15; 435/6.18; 435/6.12; 435/6.11; 435/7.4; 506/16; 506/18

(57) ABSTRACT

The invention is generally directed to a diagnostic method for predicting the benefit of the response of a subject diagnosed with cancer to a platinum compound-based adjuvant chemotherapy, preferably to a cisplatin-based chemotherapy which implements the determination of the PARP expression level in the biological sample containing tumor cells, and, preferably, together with the MSH2 and/or ERCC1 expression level, more preferably together with the MSH2 and ERCC1 expression levels.

The invention is generally directed to a diagnostic method for predicting the benefit of the response of a subject diagnosed with cancer to a platinum compound-based adjuvant chemotherapy, preferably to a cisplatin-based chemotherapy which implements the determination of the PARP expression level in the biological sample containing tumor cells, and, preferably, together with the MSH2 and/or ERCC1 expression level, more preferably together with the MSH2 and ERCC1 expression levels.

Platinum compound-based adjuvant chemotherapy, such as cisplatin, has become a new standard of care for cancer patients, particularly non-small cell lung cancer (NSCLC) patients. Platinum compounds are a hallmark of chemotherapy against lung cancer due to their DNA binding capacity which results in DNA damage and cell death. As DNA repair has a determinant role in both cancer susceptibility (high DNA repair capacity is a barrier against carcinogenesis) and drug resistance (high DNA repair capacity prevents platinum-induced DNA damage), it has been suggested to play a Janus-faced role in cancer physiology¹.

Poly-ADP ribosylation (PAR) is a posttranslational modification of proteins or histones that is catalyzed by the poly-ADP ribose polymerase 1 (PARP1) and PARP2. The PAR process is one of the earliest cellular responses to DNA damage. PARP1 and 2 recognize and bind to DNA damage sites and activate themselves by automodification. This process causes chromatin decondensation around the damage sites with recruitment of repair machineries such as the base excision repair (BER) complexes DNA ligase III—XRCC1. The system greatly accelerates DNA damage repair, especially in the case of single-stranded breaks (SSBs)²³. PARP1 also seems to affect double-strand break (DSB) repair, since PARP1 deficient cells are hypersensitive to DSB inducing agents. Indeed, PARP1 seems able to slow down the progression of the replication fork during Homologous Recombination-dependent DSB repair 24.

The inactivation of PARP in mice leads to increased sensitivity to ionizing radiation and alkylating agents and to genomic instability^{25, 26}. The affinity of PARP1 for platinum-modified DNA was very recently established by Guggenheim et al.²⁷. They used modified cisplatin analogues to synthesize 25 bp DNA duplexes containing platinum intrastrand crosslinks. Proteins that had affinity for these platinated DNAs included the DNA repair factors RPA1 (NER pathway), Ku70, Ku80, DNA-PKcs (NHEJ pathway), MSH2 (MMR and ICL-R pathways), DNA ligase III, PARP1 (BER pathway), as well as HMG-domain proteins HMGB1, HMGB2, HMGB3, and UBF1.

Further, since PARP operates as a modulator of BER capacity, PARP is a potential target for inhibiting DNA repair, and modulation of (ADP-ribosyl)ation is therefore considered a promising approach in clinical practice²⁸. Indeed, PARP inhibition can lead to cell death, which suggests a promising approach to sensitize tumor cells to chemo- and radiotherapy, particularly in BRCA-deficient cells which are highly sensitive to inhibition of PARP1.

MutS homolog 2 (MSH2), which is frequently mutated in hereditary nonpolyposis colon cancer, encodes a component of the mismatch repair pathway². MSH2 binds to DNA mismatches, thereby initiating DNA repair. MSH2 also binds to cisplatin-induced DNA cross-links, thereby initiating their excision and repair^{3, 4}. The loss of MSH2 may therefore impair the repair of cisplatin-induced DNA cross-links, which are highly deleterious to tumor cells. Indeed, MSH2 is required, together with critical components of other DNA damage repair pathways, such as the excision repair cross-complementing group 1 (ERCC1) protein, to repair cisplatin-induced DNA inter-strand cross-links^{5, 6}.

MSH2 expression is reduced in 10% to 58% of non-small cell lung cancer (NSCLC)⁷⁻¹⁶. A recent study reported that loss of MSH2 expression in tumors from patients with advanced NSCLC led to higher rates of response to oxaliplatin-based chemotherapy, but not to cisplatin-based chemotherapy¹⁴. In a larger study of genetic polymorphisms among patients with advanced NSCLC, the MSH2 gIV12-6T>C variant was associated with low MSH2 expression and better response to cisplatin.

The biology of the tumor is of a great interest when choosing the optimal therapy for patients with cancer, particularly for NSCLC. A number of potential biomarkers is under investigation in the hope that it will be possible to identify markers that assist in the selection of patients for specific therapies.

Promising results so far suggest that customized therapy for individual patients with the help of predictive biomarkers is possible and it is likely that this strategy will improve treatment of NSCLC in the future.

Thus, it remains desirable to provide new markers capable to predict the benefit of the response of a subject diagnosed with cancer, particularly with NSCLC, to a platinum-based chemotherapy such as cisplatin-based chemotherapy.

This is the object of the present invention.

The inventors have studied the correlation between cisplatin sensitivity and nucleoplasmic expression of PARP1 in NSCLC by immunohistochemical (IHC) assessment of the nuclear expression of PARP1 in tumors from adequate series of patients. They have demonstrated that the level of expression of PARP1 is a predictive factor of cisplatinum-based chemotherapy in lung cancer, preferably in combination with the level of expression of MSH2 and/or ERCC1 and, more preferably, in combination with the levels of expression of MSH2 and ERCC1.

In a second aspect, the inventors have demonstrated that this predicting of the benefit or not of the response of a subject diagnosed with cancer to a platinum-based chemotherapy from a biological sample from said subject can be ameliorated by using simultaneously MSH2 and/or ERCC1 marker(s) as additional predictive factor(s) of said platinum-based chemotherapy, particularly by analysing the level of expression of MSH2 and/or ERCC1 by IHC from a biological sample from said subject.

Thus, in a first aspect, the invention is directed to a method for in vitro predicting the benefit of the response of a subject diagnosed with cancer to a platinum-based chemotherapy from a biological sample from said subject comprising the following steps of:

a) determining the poly (ADP-ribose) polymerase (PARP) expression level in the biological sample; and
b) optionally, comparing said PARP expression level to the PARP expression level of a reference control or population, or compared to the non tumor cells content.

In a preferred embodiment, the method according to the present invention further comprises the following steps of:

c) determining the MSH2 and/or ERCC1 expression level(s) from the same or from another biological sample; and
d) optionally, comparing said MSH2 and/or ERCC1 expression level(s) to the MSH2 and/or ERCC1 expression level(s) of a reference control or population, or to the MSH2 and/or ERCC1 expression level(s) of non tumor cells.

According to the present invention, if the PARP expression level obtained in step a) for the patient biological sample is in step b) less or equal than the expression level obtained for said reference population, or is decreased compared to the non tumor cells, then an overall survival and/or a disease-free survival benefit can be predicted.

According to the present invention, if the PARP and the MSH2 expression levels obtained respectively in steps a) and c) for the patient biological sample are in steps b) and d) respectively less or equal than the PARP and the MSH2 expression levels obtained for said reference populations, or are decreased compared to the non tumor cells, then an overall survival benefit and/or a disease-free survival, preferably a disease-free survival can be predicted.

It is also disclosed that if the PARP and the ERCC1 expression levels obtained respectively in steps a) and c) for the patient biological sample are in steps b) and d) respectively less or equal than the PARP and the ERCC1 expression levels obtained for said reference populations, or are decreased compared to the non tumor cells, then an overall survival and/or a disease-free survival benefit can be predicted.

According to the present invention, if the PARP and the MSH2 and the ERCC1 expression levels obtained respectively in steps a) and c) for the patient biological sample are in steps b) and d) respectively less or equal than the PARP and the MSH2 and the ERCC1 expression levels obtained for said reference populations, or are decreased compared to the non tumor cells, then an overall survival and/or a disease-free survival benefit can be predicted.

In a second aspect, the invention is directed to a method for in vitro assessing whether a platinum-based chemotherapy is appropriate for a subject diagnosed with cancer from a biological sample from said subject, said method comprising the following steps of:

a) determining the PARP, and optionally the MSH2 and/or ERCC1, expression level in said biological sample; and
b) optionally, comparing said PARP, and optionally the MSH2 and/or ERCC1, expression level to the PARP, and optionally the MSH2 and/or ERCC1, of a reference control or population, or compared to the non tumor cells content,
a platinum-based chemotherapy will be determined as an appropriate chemotherapy if the PARP expression level, and, optionally, the MSH2 and/or ERCC1 expression level(s), is (are respectively) less or equal than the PARP, and optionally the MSH2 and/or ERCC1, expression level(s) of a reference control or population, or is decreased compared to the non tumor cells, and
a platinum-free chemotherapy will be determined as an appropriate chemotherapy if the PARP, and optionally the MSH2 and/or ERCC1, expression level is greater than the PARP and optionally the MSH2 and/or ERCC1, expression level of said reference control or population, or is increased compared to the non tumor cells.

In a preferred embodiment, the method of the present invention for in vitro assessing whether a platinum-based chemotherapy is appropriate for a subject diagnosed with cancer from a biological sample from said subject, is a method comprising the following steps of:

a) determining the PARP and the MSH2 expression levels in said biological sample; and
b) optionally, comparing said PARP and the MSH2 expression levels to the PARP and the MSH2 of a reference control or population, or compared to the non tumor cells content,
a platinum-based chemotherapy will be determined as an appropriate chemotherapy if the PARP expression level and the MSH2 expression level are respectively less or equal than the PARP and the MSH2 levels of a reference control or population, or is decreased compared to the non tumor cells, and/or
a platinum-free chemotherapy will be determined as an appropriate chemotherapy if the PARP and the MSH2 expression levels are greater than the PARP and the MSH2 expression levels of said reference control or population, or is increased compared to the non tumor cells.

In a more preferred embodiment, the method of the present invention for in vitro assessing whether a platinum-based chemotherapy is appropriate for a subject diagnosed with cancer from a biological sample from said subject, is a method comprising the following steps of:

a) determining the PARP, the MSH2 and the ERCC1 expression levels in said biological sample; and
b) optionally, comparing said the PARP, the MSH2 and the ERCC1 expression levels to the PARP, the MSH2 and the ERCC1 of a reference control or population, or compared to the non tumor cells content,
a platinum-based chemotherapy will be determined as an appropriate chemotherapy if the PARP, the MSH2 and the ERCC1 are respectively less or equal than the PARP, the MSH2 and the ERCC1 of a reference control or population, or is decreased compared to the non tumor cells, and/or
a platinum-free chemotherapy will be determined as an appropriate chemotherapy if the PARP, the MSH2 and the ERCC1 expression levels are greater than the PARP and the MSH2 expression levels of said reference control or population, or is increased compared to the non tumor cells.

In a third aspect, the invention is directed to an in vitro screening method for selecting a subject diagnosed from cancer for a treatment with a platinum-based chemotherapy from a biological sample from said subject, said method comprising the steps of:

a) determining the PARP expression level, and optionally the MSH2 and/or ERCC1 expression level(s), in said biological sample; and
b) comparing said PARP expression level, and optionally the MSH2 and/or ERCC1 expression level(s), to respectively the PARP expression level, and optionally the MSH2 and/or ERCC1 expression level(s), of a reference control or population, or to the PARP expression level, and optionally the MSH2 and/or ERCC1 expression level(s) contained in non tumor cells,
wherein said subject will be selected for a platinum-based chemotherapy if the PARP, expression level, and optionally the MSH2 and/or ERCC1 expression level(s) is (are respectively) less or equal than the PARP expression level, and optionally the MSH2 and/or ERCC1 expression level(s), of a reference control or population, or is decreased compared to respectively the PARP expression level, and optionally the MSH2 and/or ERCC1 expression level(s), contained in non tumor cells.

In a preferred embodiment, said in vitro screening method of the present invention for selecting a subject diagnosed from cancer for a treatment with a platinum-based chemotherapy from a biological sample from said subject, is a method comprising the steps of:

a) determining the PARP and the MSH2 expression levels, in said biological sample; and
b) comparing said PARP and MSH2 expression levels to respectively the PARP and the MSH2 expression levels of a reference control or population, or to the PARP and the MSH2 expression levels contained in non tumor cells, wherein:
said subject will be selected for a platinum-based chemotherapy if the PARP and the MSH2 expression levels are respectively less or equal than the PARP and the MSH2 expression levels of a reference control or population, or are decreased compared to respectively the PARP and the MSH2 expression levels contained in non tumor cells;
said subject will be not selected for a platinum-based chemotherapy if the PARP and the MSH2 expression levels are respectively greater than the PARP and the MSH2 expression levels of a reference control or population, or are increased compared to respectively the PARP and the MSH2 expression levels contained in non tumor cells.

In a more preferred embodiment, said in vitro screening method of the present invention for selecting a subject diagnosed from cancer for a treatment with a platinum-based chemotherapy from a biological sample from said subject, is a method comprising the steps of:

a) determining the PARP, the MSH2 and the ERCC1 expression levels, in said biological sample; and
b) comparing said PARP, MSH2 and ERCC1 expression levels to respectively the PARP, the MSH2 and the ERCC1 expression levels of a reference control or population, or to the PARP, the MSH2 and the ERCC1 expression levels contained in non tumor cells,
wherein:
said subject will be selected for a platinum-based chemotherapy if the PARP, the MSH2 and the ERCC1 are respectively less or equal than the PARP, the MSH2 and the ERCC1 expression levels of a reference control or population, or are decreased compared to respectively the PARP, the MSH2 and the ERCC1 expression levels contained in non tumor cells;
said subject will be not selected for a platinum-based chemotherapy if the PARP, the MSH2 and the ERCC1 expression levels are respectively greater than the PARP, the MSH2 and the ERCC1 expression levels of a reference control or population, or are increased compared to respectively the PARP, the MSH2 and the ERCC1 expression levels contained in non tumor cells.

Preferably, said subject is a human patient.

In a preferred embodiment, said chemotherapy is an adjuvant-platinum-based chemotherapy.

In a more preferred embodiment, said platinum-based chemotherapy or said adjuvant-platinum-based chemotherapy is selected from the group consisting of cisplatin-, carboplatin- or oxaliplatin-based chemotherapy, the most preferred being cisplatin-based chemotherapy.

In another aspect, the present invention comprises the use of PARP, preferably together with MSH2 and/or ERCC1, more preferably together with MSH2 and ERCC1 as a prognostic marker(s) associated with longer overall survival of a subject suffering from cancer.

In the method of the present invention or for the use of PARP, preferably together with MSH2 and/or ERCC1, more preferably together with MSH2 and ERCC1 as a prognostic marker(s) of the invention, the diagnosed cancer or the subject suffers from a cancer selected from the group of cancer consisting of malignant mesothelioma, bladder cancer, testicular cancer, cancer of the upper aero-digestive tract, lung cancer, triple negative breast cancer or ovarian cancer.

Preferably, when the method of the present invention implements MSH2 as an additional prognostic marker, these patients suffering from this cancer are not patients presenting the Lynch syndrome (also called hereditary nonpolyposis colorectal cancer) which is the most common hereditary colorectal cancer syndrome and often associated with mutations in the MSH2 gene or in the MLH1 gene (also related to the mismatch repair system).

In a preferred embodiment of the method, or the use of PARP, preferably together with MSH2 and/or ERCC1, more preferably together with MSH2 and ERCC1 as a prognostic marker(s), of the present invention, the diagnosed cancer is the NSCLC.

In a preferred embodiment of the method of the present invention said biological sample from the patient suffered from a cancer is a tissue sample comprising cancer cells, particularly a biopsy including tissue containing tumor cells.

Said tissue sample can be fixed, paraffin-embedded, or fresh, or frozen.

In a preferred embodiment of the method of the present invention, said PARP expression level, and optionally in step c), or where it is necessary to determine, said MSH2 and/or ERCC1 expression level(s), to be determined is (are) the level of the PARP RNA transcript, and optionally the MSH2 and/or ERCC1 RNA transcript(s), or the level of the PARP protein, and optionally the MSH2 and/or ERCC1 protein(s).

In a preferred embodiment, the PARP expression level, and optionally the MSH2 and/or ERCC1 expression level(s), is (are) determined by:
a method including a PCR or a RT-PCR method, or a Northern method when the determined PARP expression level, and optionally the determined MSH2 and/or ERCC1 expression level(s), is (are) the RNA transcript(s); or
a Western blot method or an immunohistochemistry method when the determined PARP expression level, and optionally the determined MSH2 and/or ERCC1 expression level(s), is (are) the PARP protein, and optionally the MSH2 and/or ERCC1 protein, or a specific fragment thereof.

In a preferred embodiment, in the step of determining the PARP expression level, and optionally the determined MSH2 and/or ERCC1 expression level(s), in the biological sample, the PARP expression product, and optionally the MSH2 and/or ERCC1 expression product, to be determined is (are respectively) the PARP protein, and optionally the MSH2 and/or ERCC1 protein, or a specific fragment thereof.

In a more preferred embodiment, in the step a) and c) of determining the PARP expression level, and optionally the MSH2 and/or the ERCC1 expression level(s), said biological sample is preferably a tissue sample from the tumor, and the PARP expression level, and optionally the MSH2 and/or the ERCC1 expression level(s), are level of protein expression determined by immunohistochemistry.

Preferably, when using an immunohistochemistry method on a tumor tissue sample from the subject, the PARP nuclear reactivity, and optionally the MSH2 and/or the ERCC1 nuclear reactivity is (are) determined.

Preferably reactive lung or stroma cells can be served as internal positive control and the tumor cells can be graded using the expression level in fibroblasts or endothelial cells as reference.

Preferably, the PARP protein is the human PARP protein, more preferably the human PARP1 protein, the most preferred being the human PARP1 protein having the sequence SEQ ID NO: 1 depicted under the Genbank accession number NP_{—}001609 [poly (ADP-ribose) polymerase family, member 1 [Homo sapiens]; 1014 aa linear, VERSION NP_{—}001609.2 GI:1565239681014 (28-JUN-2009)];

SEQ ID NO: 1:
MAESSDKLYRVEYAKSGRASCKKCSESIPKDSLRMAIMVQSPMFDGKVPH
WYHFSCFWKVGHSIRHPDVEVDGFSELRWDDQQKVKKTAEAGGVTGKGQD
GIGSKAEKTLGDFAAEYAKSNRSTCKGCMEKIEKGQVRLSKKMVDPEKPQ
LGMIDRWYHPGCFVKNREELGFRPEYSASQLKGFSLLATEDKEALKKQLP
GVKSEGKRKGDEVDGVDEVAKKKSKKEKDKDSKLEKALKAQNDLIWNIKD
ELKKVCSTNDLKELLIFNKQQVPSGESAILDRVADGMVFGALLPCEECSG
QLVFKSDAYYCTGDVTAWTKCMVKTQTPNRKEWVTPKEFREISYLKKLKV
KKQDRIFPPETSASVAATPPPSTASAPAAVNSSASADKPLSNMKILTLGK
LSRNKDEVKAMIEKLGGKLTGTANKASLCISTKKEVEKMNKKMEEVKEAN
IRVVSEDFLQDVSASTKSLQELFLAHILSPWGAEVKAEPVEVVAPRGKSG
AALSKKSKGQVKEEGINKSEKRMKLTLKGGAAVDPDSGLEHSAHVLEKGG
KVFSATLGLVDIVKGTNSYYKLQLLEDDKENRYWIFRSWGRVGTVIGSNK
LEQMPSKEDAIEHFMKLYEEKTGNAWHSKNFTKYPKKFYPLEIDYGQDEE
AVKKLTVNPGTKSKLPKPVQDLIKMIFDVESMKKAMVEYEIDLQKMPLGK
LSKRQIQAAYSILSEVQQAVSQGSSDSQILDLSNRFYTLIPHDFGMKKPP
LLNNADSVQAKVEMLDNLLDIEVAYSLLRGGSDDSSKDPIDVNYEKLKTD
IKVVDRDSEEAEIIRKYVKNTHATTHNAYDLEVIDIFKIEREGECQRYKP
FKQLHNRRLLWHGSRTTNFAGILSQGLRIAPPEAPVTGYMFGKGIYFADM
VSKSANYCHTSQGDPIGLILLGEVALGNMYELKHASHISKLPKGKHSVKG
LGKTTPDPSANISLDGVDVPLGTGISSGVNDTSLLYNEYIVYDIAQVNLK
YLLKLKFNFKTSLW.

Preferably, the PARP mRNA is the human PARP mRNA, more preferably the human PARP-1 mRNA, the most preferred being the human PARP-1 mRNA having the sequence SEQ ID NO: 2 depicted under the Genbank accession number NM_{—}001618. [Homo sapiens poly (ADP-ribose) polymerase 1 (PARP1), mRNA, 4001 bp mRNA linear, VERSION NM_{—}001618.3 GI:156523967;

SEQ ID NO: 2:
AGGCATCAGCAATCTATCAGGGAACGGCGGTGGCCGGTGCGGCGTGTT
CGGTGGCGGCTCTGGCCGCTCAGGCGCCTGCGGCTGGGTGAGCGCACG
CGAGGCGGCGAGGCGGCAGCGTGTTTCTAGGTCGTGGCGTCGGGCTTC
CGGAGCTTTGGCGGCAGCTAGGGGAGGATGGCGGAGTCTTCGGATAAG
CTCTATCGAGTCGAGTACGCCAAGAGCGGGCGCGCCTCTTGCAAGAAA
TGCAGCGAGAGCATCCCCAAGGACTCGCTCCGGATGGCCATCATGGTG
CAGTCGCCCATGTTTGATGGAAAAGTCCCACACTGGTACCACTTCTCC
TGCTTCTGGAAGGTGGGCCACTCCATCCGGCACCCTGACGTTGAGGTG
GATGGGTTCTCTGAGCTTCGGTGGGATGACCAGCAGAAAGTCAAGAAG
ACAGCGGAAGCTGGAGGAGTGACAGGCAAAGGCCAGGATGGAATTGGT
AGCAAGGCAGAGAAGACTCTGGGTGACTTTGCAGCAGAGTATGCCAAG
TCCAACAGAAGTACGTGCAAGGGGTGTATGGAGAAGATAGAAAAGGGC
CAGGTGCGCCTGTCCAAGAAGATGGTGGACCCGGAGAAGCCACAGCTA
GGCATGATTGACCGCTGGTACCATCCAGGCTGCTTTGTCAAGAACAGG
GAGGAGCTGGGTTTCCGGCCCGAGTACAGTGCGAGTCAGCTCAAGGGC
TTCAGCCTCCTTGCTACAGAGGATAAAGAAGCCCTGAAGAAGCAGCTC
CCAGGAGTCAAGAGTGAAGGAAAGAGAAAAGGCGATGAGGTGGATGGA
GTGGATGAAGTGGCGAAGAAGAAATCTAAAAAAGAAAAAGACAAGGAT
AGTAAGCTTGAAAAAGCCCTAAAGGCTCAGAACGACCTGATCTGGAAC
ATCAAGGACGAGCTAAAGAAAGTGTGTTCAACTAATGACCTGAAGGAG
CTACTCATCTTCAACAAGCAGCAAGTGCCTTCTGGGGAGTCGGCGATC
TTGGACCGAGTAGCTGATGGCATGGTGTTCGGTGCCCTCCTTCCCTGC
GAGGAATGCTCGGGTCAGCTGGTCTTCAAGAGCGATGCCTATTACTGC
ACTGGGGACGTCACTGCCTGGACCAAGTGTATGGTCAAGACACAGACA
CCCAACCGGAAGGAGTGGGTAACCCCAAAGGAATTCCGAGAAATCTCT
TACCTCAAGAAATTGAAGGTTAAAAAACAGGACCGTATATTCCCCCCA
GAAACCAGCGCCTCCGTGGCGGCCACGCCTCCGCCCTCCACAGCCTCG
GCTCCTGCTGCTGTGAACTCCTCTGCTTCAGCAGATAAGCCATTATCC
AACATGAAGATCCTGACTCTCGGGAAGCTGTCCCGGAACAAGGATGAA
GTGAAGGCCATGATTGAGAAACTCGGGGGGAAGTTGACGGGGACGGCC
AACAAGGCTTCCCTGTGCATCAGCACCAAAAAGGAGGTGGAAAAGATG
AATAAGAAGATGGAGGAAGTAAAGGAAGCCAACATCCGAGTTGTGTCT
GAGGACTTCCTCCAGGACGTCTCCGCCTCCACCAAGAGCCTTCAGGAG
TTGTTCTTAGCGCACATCTTGTCCCCTTGGGGGGCAGAGGTGAAGGCA
GAGCCTGTTGAAGTTGTGGCCCCAAGAGGGAAGTCAGGGGCTGCGCTC
TCCAAAAAAAGCAAGGGCCAGGTCAAGGAGGAAGGTATCAACAAATCT
GAAAAGAGAATGAAATTAACTCTTAAAGGAGGAGCAGCTGTGGATCCT
GATTCTGGACTGGAACACTCTGCGCATGTCCTGGAGAAAGGTGGGAAG
GTCTTCAGTGCCACCCTTGGCCTGGTGGACATCGTTAAAGGAACCAAC
TCCTACTACAAGCTGCAGCTTCTGGAGGACGACAAGGAAAACAGGTAT
TGGATATTCAGGTCCTGGGGCCGTGTGGGTACGGTGATCGGTAGCAAC
AAACTGGAACAGATGCCGTCCAAGGAGGATGCCATTGAGCACTTCATG
AAATTATATGAAGAAAAAACCGGGAACGCTTGGCACTCCAAAAATTTC
ACGAAGTATCCCAAAAAGTTCTACCCCCTGGAGATTGACTATGGCCAG
GATGAAGAGGCAGTGAAGAAGCTGACAGTAAATCCTGGCACCAAGTCC
AAGCTCCCCAAGCCAGTTCAGGACCTCATCAAGATGATCTTTGATGTG
GAAAGTATGAAGAAAGCCATGGTGGAGTATGAGATCGACCTTCAGAAG
ATGCCCTTGGGGAAGCTGAGCAAAAGGCAGATCCAGGCCGCATACTCC
ATCCTCAGTGAGGTCCAGCAGGCGGTGTCTCAGGGCAGCAGCGACTCT
CAGATCCTGGATCTCTCAAATCGCTTTTACACCCTGATCCCCCACGAC
TTTGGGATGAAGAAGCCTCCGCTCCTGAACAATGCAGACAGTGTGCAG
GCCAAGGTGGAAATGCTTGACAACCTGCTGGACATCGAGGTGGCCTAC
AGTCTGCTCAGGGGAGGGTCTGATGATAGCAGCAAGGATCCCATCGAT
GTCAACTATGAGAAGCTCAAAACTGACATTAAGGTGGTTGACAGAGAT
TCTGAAGAAGCCGAGATCATCAGGAAGTATGTTAAGAACACTCATGCA
ACCACACACAATGCGTATGACTTGGAAGTCATCGATATCTTTAAGATA
GAGCGTGAAGGCGAATGCCAGCGTTACAAGCCCTTTAAGCAGCTTCAT
AACCGAAGATTGCTGTGGCACGGGTCCAGGACCACCAACTTTGCTGGG
ATCCTGTCCCAGGGTCTTCGGATAGCCCCGCCTGAAGCGCCCGTGACA
GGCTACATGTTTGGTAAAGGGATCTATTTCGCTGACATGGTCTCCAAG
AGTGCCAACTACTGCCATACGTCTCAGGGAGACCCAATAGGCTTAATC
CTGTTGGGAGAAGTTGCCCTTGGAAACATGTATGAACTGAAGCACGCT
TCACATATCAGCAAGTTACCCAAGGGCAAGCACAGTGTCAAAGGTTTG
GGCAAAACTACCCCTGATCCTTCAGCTAACATTAGTCTGGATGGTGTA
GACGTTCCTCTTGGGACCGGGATTTCATCTGGTGTGAATGACACCTCT
CTACTATATAACGAGTACATTGTCTATGATATTGCTCAGGTAAATCTG
AAGTATCTGCTGAAACTGAAATTCAATTTTAAGACCTCCCTGTGGTAA
TTGGGAGAGGTAGCCGAGTCACACCCGGTGGCTCTGGTATGAATTCAC
CCGAAGCGCTTCTGCACCAACTCACCTGGCCGCTAAGTTGCTGATGGG
TAGTACCTGTACTAAACCACCTCAGAAAGGATTTTACAGAAACGTGTT
AAAGGTTTTCTCTAACTTCTCAAGTCCCTTGTTTTGTGTTGTGTCTGT
GGGGAGGGGTTGTTTTGGGGTTGTTTTTGTTTTTTCTTGCCAGGTAGA
TAAAACTGACATAGAGAAAAGGCTGGAGAGAGATTCTGTTGCATAGAC
TAGTCCTATGGAAAAAACCAAGCTTCGTTAGAATGTCTGCCTTACTGG
TTTCCCCAGGGAAGGAAAAATACACTTCCACCCTTTTTTCTAAGTGTT
CGTCTTTAGTTTTGATTTTGGAAAGATGTTAAGCATTTATTTTTAGTT
AAAAATAAAAACTAATTTCATACTATTTAGATTTTCTTTTTTATCTTG
CACTTATTGTCCCCTTTTTAGTTTTTTTTGTTTGCCTCTTGTGGTGAG
GGGTGTGGGAAGACCAAAGGAAGGAACGCTAACAATTTCTCATACTTA
GAAACAAAAAGAGCTTTCCTTCTCCAGGAATACTGAACATGGGAGCTC
TTGAAATATGTAGTATTAAAAGTTGCATTTGAAATTCTTGACTTTCTT
ATGGGCACTTTTGTCTTCCAAATTAAAACTCTACCACAAATATACTTA
CCCAAGGGCTAATAGTAATACTCGATTAAAAATGCAGATGCCTTCTCT
AAAAAAAAAAAAAAAAA

Preferably, the sequences of the set of primers used for RT-PCR assessment using TaqMan gene expression assays (Applied Biosystem) are the following (Tapia-Páez et al, FASEB J, 2008):

PARP1 (forward primer):
(SEQ ID NO: 3)
5′-CCCAAAGGAATTCCG AGAAA-3′,
and
PARP1 (reward primer):
(SEQ ID NO: 4)
5′-TCCTTTTTGGTGCTGATG-3′.

In a more preferred embodiment, in the step a) of the method of the present invention, the determination of the PARP protein level, is carried out by IHC on paraffin-embedded samples.

More preferably an anti-PARP antibody is used which has been recommended for detection of full-length PARP1 of human origin references of commercially available PARP1 antibodies, particularly selected from the group consisting of:
PARP-1 monoclonal (clone PAR01) antibody from Lab Vision, Ref. Cat#MS-1109 (Nosho et al., Eur J. Cancer. 2006 September; 42(14):2374-81),
PARP antibody from SIGMA Ref: 33-3100,
PARP, clone A6.4.12 from Millipore, Ref. MAB3217,
PARP1 Clone 3a46 from LifeSpan BioSciences, Ref. LS-C17218-100,
PARP1, Clone 5g186 from LifeSpan BioSciences, Ref. LS-C17206-250, and
PARP1 Polyclonal Antibody, from Novus Biologicals, NBP1-03181.

Other anti-PARP antibody references can be found on the website:

http://www.alzforum.org/res/com/ant/default.asp?antigenID=52

In a preferred embodiment, ERCC1-specific immunostaining is carried out as depicted in Olaussen et al. 2006⁽¹⁸⁾, see also Olaussen et al., 2007 (New England Journal of Medecine, 2007, ERCC1-Specific Immunostaining in Non-Small-Cell Lung Cancer. Volume 357, Number 15:1559-1561).

In a preferred embodiment, the specific anti MSH2 and the anti-ERCC1 antibody used for the immunohistochemistry method, or comprised into the kit of the present invention, are directed specifically respectively against the MSH2 and the ERCC1 human protein selected from the group consisting of the proteins having the following reference sequences: (Kamal et al, 2010⁽²⁹⁾)
DNA mismatch repair protein MSH2 (MutS protein homolog 2) (UniProtKB: P43246, SEQ ID NO: 5);
Excision repair cross-complementing 1 isoform 1 (GenBank NP 973730, SEQ ID NO: 6); and
Excision repair cross-complementing 1 isoform 2 (UniProtKB: P07992, SEQ ID NO: 7).

In a more preferred embodiment, for ERCC1, the kit of the present invention or the step of immunostaining or immunohistochemistry analysis in the method of the present invention is carried out using anti-ERCC1 monoclonal or polyclonal antibody, monoclonal being the most preferred, obtained by using the full length ERCC1 protein, isoform 1 or 2, as antigen for immunization.

In an also more preferred embodiment, for ERCC1, said kit or immunostaining or immunohistochemistry analysis is carried out using anti-ERCC1 antibody directed against a common epitope of the isoform 1 and 2 sequence (located in the consensus protein domain), the resulting antibody being able to recognize the ERCC1 isoform 1 and 2 protein.

In the most preferred embodiment, the kit of the present invention or the step of ERCC1-specific immunostaining of the method of the present invention is carried out by using a monoclonal antibody specifically directed against the full length recombinant human ERCC1 protein isoform 2.

In an also preferred embodiment, in the step a) or c) of determining the PARP expression level, and optionally the MSH2 and/or the ERCC1 expression level(s), in the biological sample, the PARP expression product, and optionally the MSH2 and/or the ERCC1 expression product(s), is (are respectively) the PARP mRNA, and optionally the MSH2 and/or the ERCC1 mRNAs, or a specific fragment thereof.

Thus, the determination of the PARP mRNA expression level, and optionally the mRNA MSH2 and/or the ERCC1 expression level(s), can be carried out by a method which comprises the following steps:

A) extraction of the total RNAs of said biological sample, preferably from tumor cells, followed, where appropriate, by purification of the mRNAs;

B) reverse transcription of the RNAs extracted in step A) via an oligo dT primer; and

C) PCR amplification of the cDNAs obtained in step B) using at least a pair of primers specific for the PARP mRNA, and optionally specific for the MSH2 mRNA and/or the ERCC1 mRNA, to be quantified.

In a preferred embodiment, when the determination of the MSH2 and/or the ERCC1 mRNA is carried out by a method comprising a PCR or RT-PCR amplification or in the kit of the present invention, primers and probe set specific for the MSH2 and/or the ERCC1 mRNA to be quantified can be designed using the following reference sequences:
mRNA encoding DNA mismatch repair protein MSH2 (MutS protein homolog 2) (GenBank; NM_{—}000251; SEQ ID NO: 8);
transcript variant 1, mRNA (GenBank NM_{—}202001; SEQ ID NO: 9) encoding the excision repair cross-complementing 1 iso form 1 protein ERCC1; and transcript variant 2, mRNA (GenBank NM_{—}001983, SEQ ID NO: 10) encoding the excision repair cross-complementing 1 isoform 2 protein ERCC1.

In a more preferred embodiment, for ERCC1 mRNA expression analysis by RT-QPCR, or in the kit of the present invention, the primer and probe sets can be designed in order to result in the quantification of the presence of an amplicon which is common to the transcript variant 1 and 2 sequence (preferably located in the consensus domain).

In the most preferred embodiment, for ERCC1 mRNA expression analysis by RT-QPCR, or in the kit of the present invention, the primer and probe sets can be designed in order to result to the quantification of the presence of a fragment amplicon comprised in the mRNA sequence encoding the full length recombinant human ERCC1 protein iso form 2.

In another aspect, the present invention is directed to a kit or array for in vitro predicting the benefit of the response of a subject diagnosed with NSCLC to a cisplatin-based chemotherapy from a biological sample from said patient wherein said kit or array comprises a reagent for assaying PARP expression.

In a preferred embodiment, said kit or array for in vitro predicting the benefit of the response of a subject diagnosed with NSCLC to a cisplatin-based chemotherapy comprises:

a)—a probe and/or or a pair of primers that specifically hybridizes to the PARP mRNA or cDNA, or to the complementary sequence thereof; or
b) an anti-PARP antibody, optionally labeled, capable of specifically recognizing the PARP protein.

In another aspect, the present invention is directed to a kit or array wherein said kit or array comprises:
a reagent for assaying PARP expression; and
a reagent for assaying MSH2 and/or ERCC1 expression, in a biological sample from a patient, and, optionally, an instruction sheet.

In a preferred embodiment, said kit or array comprises:
a reagent for assaying PARP expression and
a reagent for assaying MSH2 expression,
   in a biological sample from a patient, and, optionally, an instruction sheet.

In a more preferred embodiment, said kit or array comprises:
a reagent for assaying PARP expression;
a reagent for assaying MSH2 expression; and
a reagent for assaying ERCC1 expression
   in a biological sample from a patient, and, optionally, an instruction sheet.

In a preferred embodiment, in the kit or array according to the present invention,
the reagent for assaying the PARP expression level, and
the reagent for assaying the MSH2 expression level, and, optionally, ERCC1 expression levels,
   comprises reagents selected from the group consisting of:
   a)—a probe and/or or a pair of primers that specifically hybridizes to the PARP mRNA or cDNA, or to the complementary sequence thereof; and
i)—a probe and/or or a pair of primers that specifically hybridizes to the MSH2 mRNA or cDNA, or to the complementary sequence thereof; and/or
ii)—a probe and/or or a pair of primers that specifically hybridizes to the ERCC1 mRNA or cDNA, or to the complementary sequence thereof; or
   b) an anti-PARP antibody, optionally labelled, capable of specifically recognizing the PARP protein; and
i)—an anti-MSH2 antibody, optionally labelled, capable of specifically recognizing the MSH2 protein; and/or
ii)—an anti-ERCC1 antibody, optionally labelled, capable of specifically recognizing the ERCC1 protein.

In the method, use and kit or array according to the present invention, it is more preferred that said PARP is the poly (ADP-ribose) polymerase 1 (PARP1).

In a preferred embodiment, in the kit or array according to the present invention, the reagent for assaying MSH2 and the reagent for assaying ERCC1 expression comprises a reagent selected from the group consisting of:
a probe or a pair of primers that hybridizes specifically to the MSH2 and a probe or a pair of primers that hybridizes specifically to the ERCC1 mRNA or cDNA; or
an anti-MSH2 and an anti-ERCC1 antibody for performing a Western blot or immunohistochemistry assay.

In a preferred embodiment, said kit or array according to the present invention is for in vitro predicting the benefit of the response of a subject diagnosed with cancer to a a platinum-based chemotherapy, more preferably for in vitro predicting the benefit of the response of a subject diagnosed NSCLC with cisplatin-based chemotherapy.

In a more preferred embodiment, said kit or array according to the present invention is for in vitro predicting the non-therapeutic effect of a platinum-based chemotherapy treatment for a subject diagnosed with cancer, preferably for a subject diagnosed with NSCLC cancer with cisplatin-based chemotherapy (non-appropriate chemotherapy).

Western blotting or immunohistochemistry method can be used for analysing or quantifying specific protein expression in biological sample. Such methods are well known from the skilled man.

The blots can be detected using antibodies specifically directed against different specific regions or epitope of PARP, MSH2 or ERCC1 protein, particularly against human PARP, MSH2 or ERCC1 protein.

The term “antibody” as used herein refers to immunoglobulin molecules and immunologically active portions of immunoglobulin molecules, i.e., molecules that contain an antigen binding site which specifically binds (immunoreacts with) the PARP, MSH2 or the ERCC1 protein. The term “antibody” comprises monoclonal or polyclonal antibodies but also chimeric or humanized antibodies.

An isolated PARP, MSH2 or ERCC1 protein, such as recombinant protein, or a specific fragment thereof can be used as an immunogen to generate antibodies that bind such protein using standard techniques for polyclonal and monoclonal antibody preparation. It may be also possible to use any fragment of these protein which contains at least one antigenic determinant may be used to generate these specific antibodies.

A protein immunogen typically is used to prepare antibodies by immunizing a suitable subject (e.g., rabbit, goat, mouse or other mammal) with the immunogen. An appropriate immunogenic preparation can contain said protein, or fragment thereof, and further can include an adjuvant, such as Freund's complete or incomplete adjuvant, or similar immunostimulatory agent.

Thus, antibody for use in accordance with the invention include either polyclonal, monoclonal chimeric or humanized antibodies able to selectively bind, or which selectively bind to an epitope-containing a polypeptide comprising a contiguous span of at least 8 to 10 amino acids of an amino acid sequence of the PARP, MSH2 or the ERCC1 protein, preferably the human protein.

In a preferred embodiment, quantitative RT-PCR analysis can be used to measure the mRNA expression levels of PARP, MSH2 and ERCC1 in a biological sample or tissue sample. Gene expression analysis by real-time quantitative PCR(RT-QPCR) is well known from the skilled person.

For example PARP, MSH2 and ERCC1 mRNA expression analysis by RT-QPCR can be assessed after standard tissue sample RNA extraction (for example the samples are lysed in a tris-chloride, EDTA, sodium dodecyl sulphate and proteinase K containing buffer and RNA is then extracted with phenol-chloroform-isoamyl alcohol followed by precipitation with isopropanol in the presence of glycogen and sodium acetate). RNA is then resuspended in diethyl pyrocarbonate water (Ambion Inc., Austin, Tex.) and treated with DNAse I (Ambion Inc., Austin, Tex.) to avoid DNA contamination. Complementary DNA was synthesized using for example Maloney Murine Leukemia Virus retrotranscriptase enzyme. Template cDNA was added to Taqman Universal Master Mix (AB, Applied Biosystems, Foster City, Calif.) in a 12.5-μl reaction with specific primers and probe for each gene. The primer and probe sets can be designed using Primer Express 2.0 Software (AB) and the reference sequences (which can be obtained on the web site http://www.ncbi.nlm.nih.gov/entrez/query.fcgi?db=gene). Primers and probe for the ERCC1 mRNA expression analysis by RT-QPCR can be:
ERCC1 (reference Genbank sequence NM_{—}001983 3-4, Exon Boundary 3-4) (Bellmunt et al., Annals of Oncology 2007 18(3):522-528).

Forward primer
(SEQ ID NO: 11)
5′ GGG AAT TTG GCG ACG TAA TTC 3′;
Reward primer:
(SEQ ID NO: 12)
5′ GCG GAG GCT GAG GAA CAG 3′;
and
Labeled Probe 6FAM
(SEQ ID NO: 13)
5′ CAC AGG TGC TCT GGC CCA GCA CAT A 3′ TAMRA.

MSH2: labeled probe-mix from Applied Biosystems can be used (Helleman et al., BMC Cancer. 2006; 6:201);
Applied Biosystems, TaqMan® Gene Expression Assays, Assay ID Hs00179887 ml (Reference Sequence: GenBank NM_{—}000251.1, Translated protein: NP_{—}000242.1 Exon Boundary: 14-15; location 2529; Amplicon Length: 87).

Quantification of gene expression was carried out using the ABI Prism 7900HT Sequence Detection System (AB).

A preferred agent for detecting and quantifying mRNA or cDNA encoding the PARP, MSH2 or the ERCC1 protein, is a labeled nucleic acid probe or primers able to hybridize this mRNA or cDNA. The nucleic acid probe can be an oligonucleotide of at least 10, 15, 30, 50 or 100 nucleotides in length and sufficient to specifically hybridize under stringent conditions to the mRNA or cDNA. The nucleic acid primer can be an oligonucleotide of at least 10, 15 or 20 nucleotides in length and sufficient to specifically hybridize under stringent conditions to the mRNA or cDNA, or complementary sequence thereof (preferred are oligonucleotide primers or probe having at least 90%, 95%, 99% and 100% identity with the mRNA sequence fragment or the complementary sequence thereof).

A preferred agent for detecting and quantifying the PARP, MSH2 or the ERCC1 protein, is an antibody able to bind specifically to this protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

For example, in vitro techniques for detection of candidate mRNA include Northern hybridizations and in situ hybridizations. In vitro techniques for detection of the candidate protein include enzyme linked immunosorbent assays (ELISAs), Western blots, immunoprecipitations and immunofluorescence. In vitro techniques for detection of candidate cDNA include Southern hybridizations.

When the invention encompasses kits for quantifying the level of the PARP, and the level of MSH2 and/or the ERCC1 protein, the kit can comprise a labeled compound or agent capable of quantifying these proteins. Said agents can be packaged in a suitable container. The kit can further comprise instructions for using the kit to quantify the level of the PARP protein, and the MSH2 and/or the ERCC1 protein, or of the PARP mRNA, and the MSH2 and/or the ERCC1 mRNA(s).

In certain embodiments of the method of the present invention, the determination of the PARP transcript (mRNA), and the MSH2 and/or the ERCC1 transcript(s), involves the use of a probe/primer in a polymerase chain reaction (PCR), such as anchor PCR or RACE PCR, or, alternatively, in a ligation chain reaction (LCR), or alternatively quantitative real time RT-PCR. This method can include the steps of collecting a sample of tumor cells from a patient, isolating nucleic acid (e.g. mRNA) from the tumor cells of the sample, optionally transforming mRNA into corresponding cDNA, contacting the nucleic acid sample with one or more primers which specifically hybridize to the PARP, MSH2 or the ERCC1 mRNA or their corresponding cDNA under conditions such that hybridization and amplification of the PARP, MSH2 or the ERCC1 mRNA or cDNA occurs, and quantifying the presence of the amplification products. It is anticipated that PCR and/or LCR may be desirable to use as an amplification step in conjunction with any of the techniques used for quantifying nucleic acid detecting.

The methods described herein may be performed, for example, by utilizing pre-packaged diagnostic kits comprising at least one probe nucleic acid or set of primer or antibody reagent described herein, which may be conveniently used, e.g., in clinical settings to follow-up or diagnose patients.

The following examples and also the figures and the legends hereinafter have been chosen to provide those skilled in the art with a complete description in order to be able to implement and use the present invention. These examples are not intended to limit the scope of what the inventor considers to be its invention, nor are they intended to show that only the experiments hereinafter were carried out.

### LEGENDS OF THE FIGURES

FIGS. 1A and 1B: Examples of MSH2 positive and MSH2 negative non-small cell lung cancer.

FIG. 1A: MSH2 positive case. The staining is intense and diffuse.

FIG. 1B: MSH2 negative case. The arrow shows positive stromal cells (internal control).

FIGS. 2A-2F: Kaplan-Meier estimates of the probability of overall survival

FIG. 2A: Patients with MSH2 negative tumors

FIG. 2B: Patients with MSH2 positive tumors

FIG. 2C: Patients with MSH2 negative/ERCC1 negative tumors

FIG. 2D: Patients with MSH2 positive/ERCC1 positive tumors

FIG. 2E: Patients with MSH2 negative/ERCC1 positive tumors

FIG. 2F: Patients with MSH2 positive/ERCC1 negative tumors.

FIGS. 3A-3D: Examples of PARP positive and PARP negative non-small cell lung cancer.

FIG. 3A: PARP negative case (X20).

FIG. 3B: PARP negative case (X400).

FIG. 3C: PARP positive case. (X20).

FIG. 3D: PARP positive case. (X400).

FIGS. 4A-4D: Kaplan-Meier estimates of the prognostic value of the 3 positive markers on the overall survival and on the disease-free survival

FIG. 4A: Overall Survival in control group

FIG. 4B: Disease-Free Survival in control group

FIG. 4C: Overall Survival in chemotherapy group

FIG. 4D: Disease-Free Survival in chemotherapy group

FIGS. 5A-5B: Kaplan-Meier estimates of the predictive value of the 3 positive markers on the overall survival and on the disease-free survival

FIG. 5A: Overall Survival

FIG. 5B: Disease-Free Survival

### EXAMPLES

### Material and Methods

A) Patients and study design

All patients had participated in the IALT study (1867 patients)¹⁷. The IALT-Bio study was designed to examine whether markers predicted survival due to chemotherapy¹⁸. The 822 patients who were treated in centres that recruited fewer than 10 patients in the clinical IALT study were excluded from the biological study to facilitate specimen collection and to limit the “centre-effect” on the subsequent biomarker analysis. After tissue collection and a pathological review, 783 tissue blocks were included in the IALT-Bio study.

### B) Tissue Micro-Array (TMA) Construction

Three representative tumor areas were selected for each case. Cores measuring 0.6 mm in diameter and 5 mm in length (spots) were arrayed following a map. Among the 783 IALT-Bio tissue blocks that contained tumor material, triplicate spots were obtained in 768 (98%) cases. Random spots were compared to the original blocks to verify agreement between their coordinates and the original samples.

### C) PARP Immunostaining and its Evaluation

Immunostaining was performed following a standard procedure using the Vectastain Elite kit with NovaRED (Vector Laboratories, Burlingame, Calif., USA) as the substrate and Mayer's haematoxylin as the counterstain. The primary antibody was for example, the anti-PARP1 monoclonal antibody (clone PAR01) from Lab Vision, Ref Cat#MS-1109 raised against the human PARP1. For epitope retrieval, slides were heated at 98° C. for one hour in 10 mM citrate buffer, pH 7.3. Sections were incubated at room temperature for 90 minutes with the PAR01 anti-PARP1 diluted monoclonal antibody. The freshly cut TMA sections were manually immunostained in a single experiment that included a tonsil section as an external control.

Two investigators who were blinded to clinical data, independently evaluated PARP nuclear reactivity. The TMA slides were scanned at high resolution (VM3 virtual scanner, Ziemens, Germany), enabling the study of an identical high-quality image at 20× magnification for each spot for detailed evaluation.

The spots were carefully examined for reactive lung or stromal cells (endothelial cells and fibroblasts), which served as an internal positive control. Spots without a valid internal control were discarded. Cases for which no valid tumor spot could be evaluated were excluded.

Staining intensity was graded on a scale of 0-3, using the expression level in fibroblasts or endothelial cells as a reference (defined a priori as a score of 2). The percentage of reactive tumor cells was graded on a scale of 0%-100%. A proportion score was assigned to the percentage of reactive tumor nuclei: 0 was assigned if 0% of reactive tumor nuclei were found, 0.1 if 1%-9%, 0.5 if 10%-49%, and 1 if >50% of reactive tumor nuclei were present. This proportion score was multiplied by the staining intensity to obtain a histology score (H-score) for each patient'^{s}. All discordant cases were reviewed in order to reach a consensus.

### D) MSH2 Immunostaining and its Evaluation

Immunostaining was performed following a standard procedure using the Vectastain Elite kit with NovaRED (Vector Laboratories, Burlingame, Calif., USA) as the substrate and Mayer's haematoxylin as the counterstain. The primary antibody was the mouse monoclonal antibody FE11 (Calbiochem, San Diego, Calif., USA) raised against the C-terminal fragment of human MSH2. For epitope retrieval, slides were heated at 98° C. for one hour in 10 mM citrate buffer, pH 7.3. Sections were incubated at room temperature for 90 minutes with the FE11 antibody at a dilution of 1:50. The freshly cut TMA sections were manually immunostained in a single experiment that included a tonsil section as an external control (Kamal et al., 2010⁽²⁹⁾).

See Paragraph C) above for the evaluation of MSH2 immunostaining

### E) Statistical Analysis

Long-term IALT survival data were used with a median follow-up of 7.5 years¹⁹.

A logistic model stratified by centre was used to compare patients with PARP-positive and PARP-negative tumors, to compare patients with MSH2-positive and MSH2-negative tumors and to compare patients with ERCC1-positive and ERCC1-negative tumors.

The prognostic values of the biomarker status and chemotherapy for overall survival were studied using the Cox model. As in the main IALT analysis¹⁶, the Cox model included every factor used in the stratified randomisation (centre, tumor stage, and type of surgery) plus clinical and histological prognostic factors (age, sex, WHO performance status, nodal status, lymphoid infiltration, and the revised histopathological type). All other factors that were statistically related to the biomarker status in the multivariate logistic model (P<0.05) were added to the Cox model.

The predictive value of the biomarker was studied by testing the interaction between the biomarker status and the allocated treatment (chemotherapy versus observation) in the same Cox model. Tests of homogeneity of the hazard ratios were performed within the Cox model. All reported P values were two-sided. Survival rates were estimated using the Kaplan-Meier method (all P values indicated, besides Kaplan-Meier curves, were adjusted P values corresponding to the Cox analysis).

All analyses were performed using SAS software, version 9.1 (SAS Institute Inc. Cary N.C., USA) and curves were drawn with the Tigre™ software.

### F) ERCC1 Immunostaining

ERCC1-specific immunostaining is carried out as depicted in Olaussen et al. 2006⁽¹⁸⁾, paragraph “ERCC1 Immunostaining” page 985. The mouse monoclonal “8F1” specifically directed against the full length recombinant human ERCC1 protein being used.

### Example 1

### PARP1 Expression and Effect of Chemotherapy on Overall Survival According to PARP Expression

Overall, the effects of adjuvant chemotherapy on the PARP1-negative and positive groups were significantly different. In the PARP1-negative group, overall survival was longer in the chemotherapy arm than in the control arm. Among patients with PARP1-negative tumors, median overall survival was significantly longer in the chemotherapy arm than in the control arm.

### Example 2

### MSH2 Expression, Baseline Clinical Characteristics, Overall Survival and Adjuvant Chemotherapy, Effect of Chemotherapy on Overall Survival According to MSH2 Expression and Prognostic Effect of MSH2 Expression on Overall Survival

### a) MSH2 Expression

Among the 768 patients whose tumor was included in the TMA, no tumor material could be analysed after immunohistochemistry in 34 (4%) cases. After excluding the cases without valid internal controls, the H-scores were evaluated in 673/768 (88%) patients.

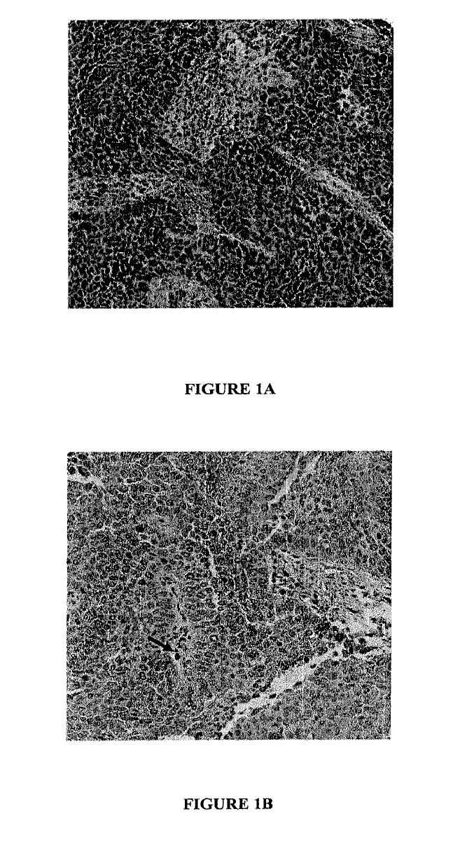

The H-scores were: 0.2 in 1 case (0%); 1 in 52 cases (8%); 2 in 363 cases (54%); and 3 in 257 cases (38%). The median H-score was 2 which was chosen to separate positive cases (H-score=3) and negative cases (H-score<3). There were 257 (38%) positive cases and 416 (62%) negative cases. FIGS. 1A-1B show examples of positive and negative cases.

### b) Baseline Clinical Characteristics

The relationships between MSH2 expression and the clinical characteristics are provided in Table 1. The proportion of adenocarcinomas was lower (P=0.002) for MSH2-positive cases (20%) than for MSH2-negative cases (37%). The morphological slide quality after HE staining was associated with MSH2 expression (P=0.01).

In a logistic model adjusted on sex and slide quality, histology differed according to MSH2 expression, with fewer adenocarcinomas among patients with MSH2-positive tumors (P=0.006).

The 673 cases included in the MSH2 analysis differed from the 95 cases excluded in terms of histology (P<0.001), type of surgery (P=0.02), and slide quality (P=0.01) (there were fewer squamous-cell carcinomas, fewer pneumonectomies, and lower staining quality in excluded cases).

TABLE 1
Patient Characteristics*

| | Patients with | | Patients with | | | | |
|---|---|---|---|---|---|---|---|
| | MSH2-Positive | | MSH2 Negative | | All Patients | | |
| | Tumors (N = 257) | | Tumors (N = 416) | | (N = 673) | | P |
| Characteristic | number | percent | number | percent | number | percent | Value† |
| Sex | | | | | | | 0.05 |
| Male | 221 | 86 | 326 | 78 | 547 | 81 | |
| Female | 36 | 14 | 90 | 22 | 126 | 19 | |
| Age | | | | | | | 0.99 |
| <55 years | 70 | 27 | 127 | 31 | 197 | 29 | |
| 55-64 years | 116 | 45 | 181 | 44 | 297 | 44 | |
| >64 years | 71 | 28 | 108 | 26 | 179 | 27 | |
| Pathological TNM stage | | | | | | | 0.79 |
| Stage I | 83 | 32 | 150 | 36 | 233 | 35 | |
| Stage II | 53 | 21 | 102 | 25 | 155 | 23 | |
| Stage III | 121 | 47 | 164 | 39 | 285 | 42 | |
| Tumor | | | | | | | 0.83 |
| T1 | 36 | 14 | 63 | 15 | 99 | 15 | |
| T2 | 145 | 56 | 263 | 63 | 408 | 61 | |
| T3 | 74 | 29 | 82 | 20 | 156 | 23 | |
| T4 | 2 | 1 | 8 | 2 | 10 | 1 | |
| Nodes | | | | | | | 0.99 |
| N0 | 122 | 47 | 188 | 45 | 310 | 46 | |
| N1 | 69 | 27 | 125 | 30 | 194 | 29 | |
| N2 | 66 | 26 | 103 | 25 | 169 | 25 | |
| Histologic type | | | | | | | <0.002 |
| Adenocarcinoma | 51 | 20 | 154 | 37 | 205 | 30 | |
| Squamous-cell carcinoma | 174 | 68 | 217 | 52 | 391 | 58 | |
| Other NSCLC | 32 | 12 | 45 | 11 | 77 | 11 | |
| Surgery | | | | | | | 0.34 |
| Pneumonectomy | 118 | 46 | 161 | 39 | 279 | 41 | |
| Lobectomy or segmentectomy | 139 | 54 | 255 | 61 | 394 | 59 | |
| Performance status score [symbol] | | | | | | | 0.37 |
| 0 | 147 | 57 | 222 | 53 | 369 | 55 | |
| 1 | 84 | 33 | 165 | 40 | 249 | 37 | |
| 2 | 26 | 10 | 29 | 7 | 55 | 8 | |
| Lymphoid infiltration | | | | | | | 0.20 |
| Not intense | 235 | 91 | 360 | 87 | 595 | 88 | |
| Intense | 22 | 9 | 56 | 13 | 78 | 12 | |
| Pleural invasion | | | | | | | 0.19 |
| No | 232 | 90 | 386 | 93 | 618 | 92 | |
| Yes | 25 | 10 | 30 | 7 | 55 | 8 | |
| Vascular invasion | | | | | | | 0.44 |
| No | 181 | 70 | 295 | 71 | 476 | 71 | |
| Yes | 76 | 30 | 121 | 29 | 197 | 29 | |
| Lymphatic invasion | | | | | | | 0.19 |
| No | 71 | 28 | 137 | 33 | 208 | 31 | |
| Yes | 186 | 72 | 279 | 67 | 465 | 69 | |
| Quality after HE staining | | | | | | | 0.01 |
| Average | 37 | 14 | 28 | 7 | 65 | 10 | |
| Good | 220 | 86 | 388 | 93 | 608 | 90 | |

*TNM denotes tumor-node-metastases. Percentages may not total 100 because of rounding.
†P values testing the difference between positive and negative tumors were calculated using logistic regression stratified on centre. [symbol]
Word Health Organization scores for performance status range from 0 to 2, with a score of 0 indicating no symptoms, 1 mild symptom, and 2 moderate symptoms.

### c) Overall Survival and Adjuvant Chemotherapy

For the group of patients included in the MSH2 analysis (673 patients), the adjusted hazard ratio for death associated with chemotherapy compared to observation was 0.88 (95% CI, 0.72 to 1.07; P=0.21). The 5-year overall survival rates were 47% (95% CI, 41% to 52%) in the chemotherapy arm, and 44% (95% CI, 39% to 49%) in the control arm. The 8-year overall survival rates were 36% (95% CI, 31% to 42%) in the chemotherapy arm, and 37% (95% CI, 32% to 43%) in the control arm.

### d) Effect of Chemotherapy on Overall Survival According to MSH2 Expression

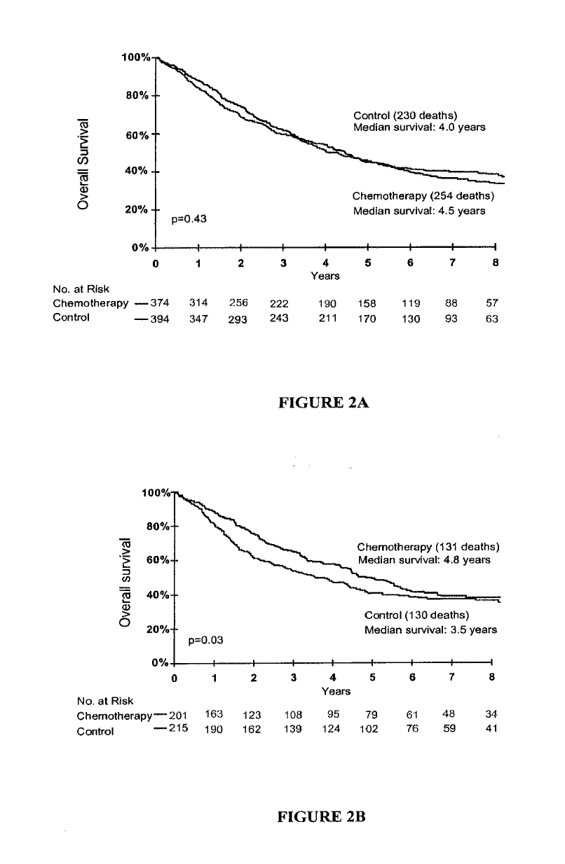

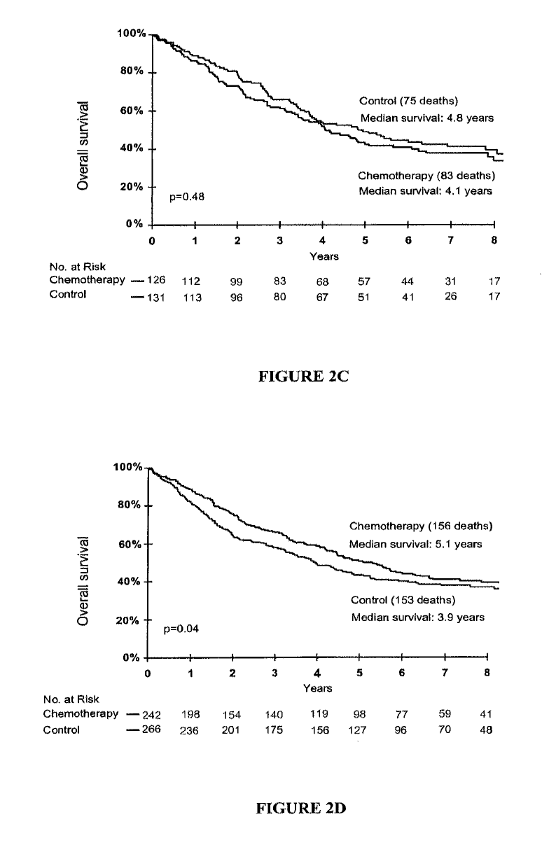

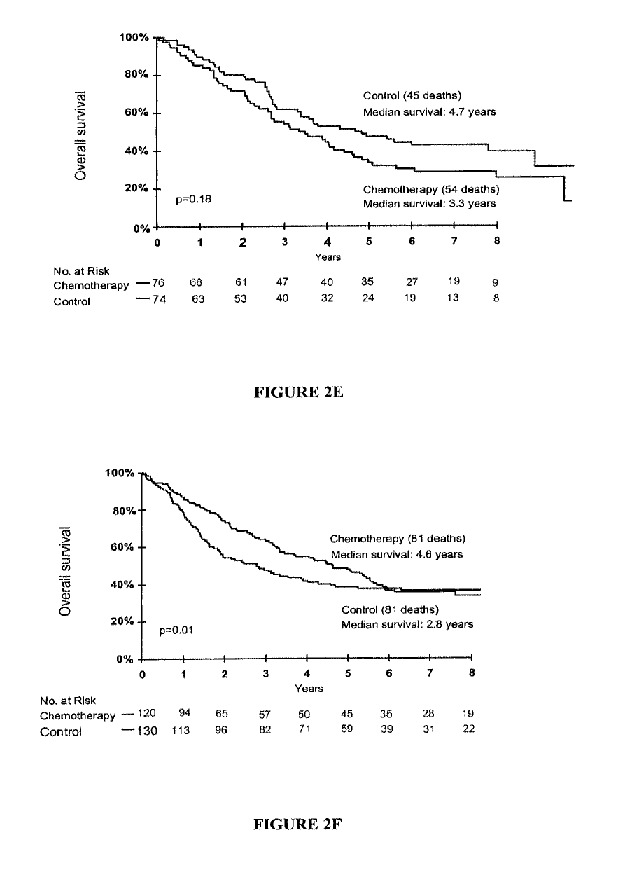

Overall, the effects of adjuvant chemotherapy on the MSH2-negative and positive groups were borderline significantly different (test for interaction, P=0.06). In the MSH2-negative group, overall survival was longer in the chemotherapy arm than in the control arm, (adjusted hazard ratio for death, 0.76; 95% CI, 0.59 to 0.97; P=0.03) (Table 2, FIG. 2A). The 5-year overall survival rates among patients with MSH2-negative tumors were 49% (95% CI, 43% to 56%) in the chemotherapy arm and 41% (95% CI, 34% to 48%) in the control arm. The 8-year overall survival rates among patients with MSH2-negative tumors were 38% (95% CI, 32% to 45%) in the chemotherapy arm and 36% (95% CI, 30% to 43%) in the control arm. Among patients with MHS2-negative tumors, median overall survival was 16 months longer in the chemotherapy arm (58 months) than in the control arm (42 months). In the MSH2-positive group, there was no difference in overall survival between the chemotherapy arm and the control arm (adjusted hazard ratio for death, 1.12; 95% CI, 0.81 to 1.55; P=0.48) (Table 2, FIG. 2B). The 5-year overall survival rates among patients with MSH2-positive tumors were 42% (95% CI, 34% to 51%) in the chemotherapy arm and 49% (95% CI, 40% to 58%) in the control arm. The 8-year overall survival rates among patients with MSH2-positive tumors were 34% (95% CI, 25% to 43%) in the chemotherapy arm and 39% (95% CI, 31% to 49%) in the control arm.

TABLE 2
Overall survival according to attributed treatment and MSH2 status

| | Chemotherapy arm | Control arm | Hazard ratio | |
|---|---|---|---|---|
| | (No deaths/No | (No deaths/No | for death | |
| | patients) | patients) | (95% CI)* | P value |
| Patients with MSH2-negative tumors | 131/215 | 130/201 | 0.75 (0.58-0.97) | p = 0.03 |
| n = 416 | | | | |
| Patients with MSH2 positive tumors | 83/131 | 75/126 | 1.12 (0.81-1.55) | p = 048 |
| n = 257 | | | | |
| Hazard ratio for death (95% CI)** | 0.99 (0.74-1.32) | 0.66 (0.49-0.90) | — | — |
| P Value | p = 0.93 | p = 0.01 | — | p = 0.06† |

*Hazard ratios are for the comparison of chemotherapy group with the control group.
**Hazard ratios are for the comparison of patients with MSH2-positive tumors with those with MSH2-negative tumors.
†The P value is for the interaction between MSH2 expression and treatment.

### e) Prognostic Effect of MSH2 Expression on Overall Survival

In the control arm, MSH2 positivity compared to MSH2 negativity was associated with an adjusted hazard ratio for death of 0.66 (95% CI, 0.49 to 0.90; P=0.01). Median overall survival was 16 months longer in the MSH2-positive group (58 months) than in the MSH2-negative group (42 months).

In the chemotherapy arm, there was no difference in overall survival between MSH2-positive and MSH2-negative tumors (adjusted hazard ratio for death, 0.99; 95% CI, 0.74 to 1.32; P=0.93).

### Example 3

### Effect of Chemotherapy on Overall Survival According to PARP, and MSH2 and/or ERCC1 Expression

### A) Effect of Chemotherapy on Overall Survival According to PARP and MSH2 Expression and Predictive Value of Combining PARP and MSH2

The expression levels of PARP and MSH2 were both available for a number of patients. In a sub-analysis of these particular patients, the effect of chemotherapy was examined in the following subgroups defined by combining PARP and MSH2 expression: PARP-positive/MSH2-positive, PARP-positive/MSH2-negative, PARP-negative/MSH2-positive, PARP-negative/MSH2-negative.

B) Effect of Chemotherapy on Overall Survival according to PARP and ERCC1 Expression and Predictive Value of combining PARP and ERCC1

The expression levels of PARP and ERCC1 were both available for a number of patients. In a sub-analysis of these particular patients, the effect of chemotherapy was examined in the following subgroups defined by combining PARP and ERCC1 expression: PARP-positive/ERCC1-positive, PARP-positive/ERCC1-negative, PARP-negative/ERCC1-positive, PARP-negative/ERCC1-negative.

### C) Effect of Chemotherapy on Overall Survival According to PARP, MSH2 and ERCC1 Expression and Predictive Value of Combining PARP, MSH2 and ERCC1

The expression levels of PARP, MSH2 and ERCC1 were together available for a number of patients. In a sub-analysis of these particular patients, the effect of chemotherapy was examined in the following subgroups defined by combining PARP, MSH2 and ERCC1 expression: PARP-positive/MSH2-positive/ERCC1-positive, PARP-positive/MSH2-positive/ERCC1-negative, PARP-positive/MSH2-negative/ERCC1-positive, PARP-positive/MSH2-negative/ERCC1-negative, PARP-negative/MSH2-positive/ERCC1-positive, PARP-negative/MSH2-positive/ERCC1-negative, PARP-negative/MSH2-negative/ERCC1-positive, and PARP-negative/MSH2-negative/ERCC1-negative.

### Example 4

### Effect of Chemotherapy on Overall Survival and on Disease-Free Survival According to PARP, MSH2 and ERCC1 Expression

### Materials and Methods

### PARP Immunostaining and its Evaluation

Immunostaining of PARP was performed on 678 whole tissue sections of the IALT-bio cohort following a standard procedure using the Vectastain ABC Elite kit with DAB (Vector Laboratories, Burlingame, Calif., USA) as the substrate and Mayer's haematoxylin as the counterstain. The primary antibody was the anti-PARP1 monoclonal antibody (PARP: cloneA6.4.12) from AbD Serotec Ref. Cat#MCA1522G raised against the human PARP1. For epitope retrieval, slides were heated at 98° C. for 30 minutes in 10 mM citrate buffer, pH 6.0. Sections were incubated at room temperature for 60 minutes with the A6.4.12 anti-PARP1 diluted (1:4000) monoclonal antibody. The freshly cut tissue sections were manually immunostained and included a tonsil section as an external control.

Two investigators who were blinded to clinical data, independently evaluated PARP nuclear reactivity under the microscope.

The tumor sections were carefully examined for PARP reactive lung or stromal cells (endothelial cells, fibroblasts, and lymphocytes), which served as an internal positive control. Cases without a valid internal control were discarded. Cases for which no valid tumor could be evaluated were excluded.

Staining intensity was graded on a scale of 0-3, using the expression level in fibroblasts or endothelial cells as a reference. The percentage of reactive tumor cells was graded on a scale of 0%-100%.

All discordant cases were reviewed in order to reach a consensus. A final histological PARP expression score was determined as the product of staining intensity and percentage of expression in tumor cells (i.e. a score graded from 0 to 300).

The median of the histological PARP expression scores was chosen as cut-off to distinguish between low (said negative) PARP expressing tumors and high (said positive) expressing tumors.

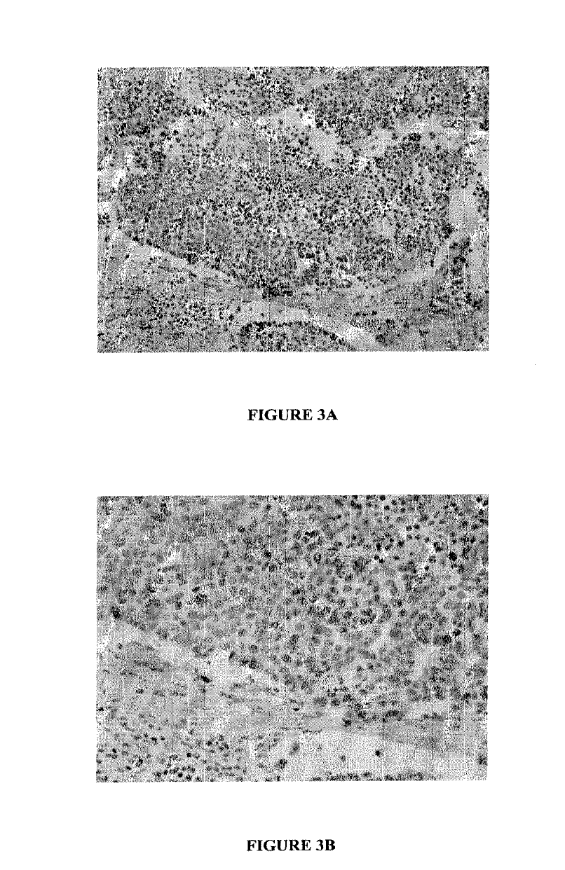

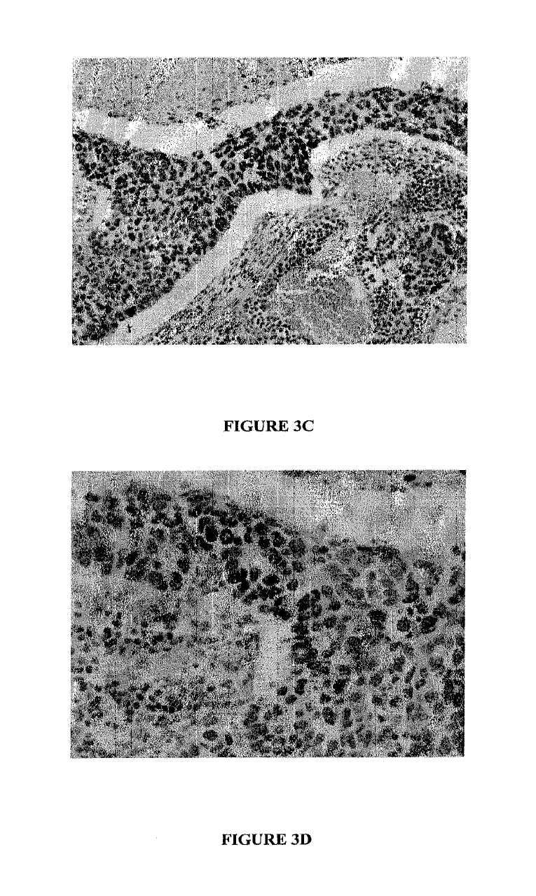

FIGS. 3A-3D show examples of positive and negative cases.

### A) Effect of Chemotherapy on Disease-Free Survival According to PARP and MSH2 Expression and Predictive Value of Combining PARP and MSH2

The expression levels of PARP and MSH2 were both available for 584 patients (86% of the patients included in IALT-Bio). In a sub-analysis of these particular patients, the effect of chemotherapy was examined in the following subgroups defined by combining PARP and MSH2 expression: PARP-positive/MSH2-positive, PARP-positive/MSH2-negative, PARP-negative/MSH2-positive, PARP-negative/MSH2-negative.

In the PARP-negative/MSH2-negative group (207 patients), the adjusted hazard ratio for death associated with chemotherapy versus observation was 0.71 (95% CI, 0.50 to 1; P=0.05) (Table 3).

TABLE 3
Disease-free survival according to allocated treatment and subgroup analysis

| | Patients with PARP - | Patients with PARP - | |
|---|---|---|---|
| | Negative tumours | Positive tumours | Total |
| Patients with MSH2-negative tumours | | | |
| Number of events/number of patients | 134/207 | 107/161 | 241/368 |
| Hazard ratio for death (95% CI) | 0.71 [0.50; 1.00] | 0.78 [0.53; 1.15] | 0.74 [0.57; 0.96] |
| P Value | p = 0.05 | p = 0.20 | p = 0.02 |
| Patients with MSH2-positive tumours | | | |
| Number of events/number of patients | 65/92 | 75/124 | 140/216 |
| Hazard ratio for death (95% CI) | 1.16 [0.71; 1.92] | 1.34 [0.85; 2.13] | 1.26 [0.90; 1.76] |
| P Value | p = 0.55 | p = 0.21 | p = 0.18 |
| Total | | | |
| Number of death/number of patients | 199/299 | 182/285 | |
| Hazard ratio for death (95% CI) | 0.83 [0.63; 1.11] | 1.01 [0.75; 1.36] | |
| P Value | p = 0.21 | p = 0.94 | |

### B) Prognostic Effect of Combining PARP, MSH2 and ERCC1 Expression on Overall and Disease-Free Survival

The expression levels of PARP, MSH2 and ERCC1 were together available for 570 patients. In a sub-analysis of these particular patients, the survival in the control arm and in the chemotherapy arm was examined in the following subgroups defined by combining PARP, MSH2 and ERCC1 expression:
0 positive marker (PARP-negative/MSH2-negative/ERCC1-negative)
1 positive marker
2 positive markers and
3 positive markers (PARP-positive/MSH2-positive/ERCC1-positive).

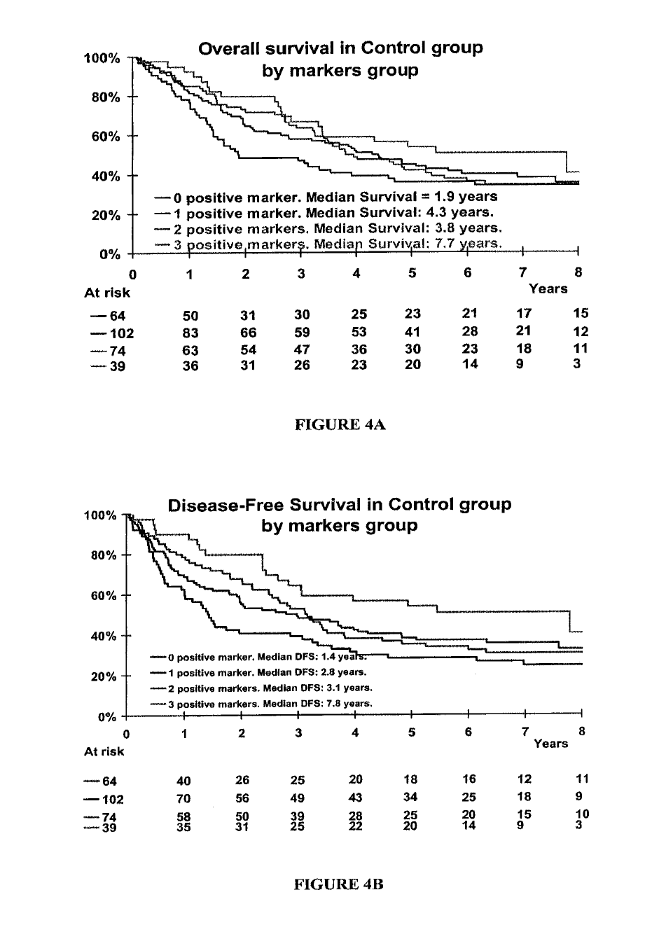

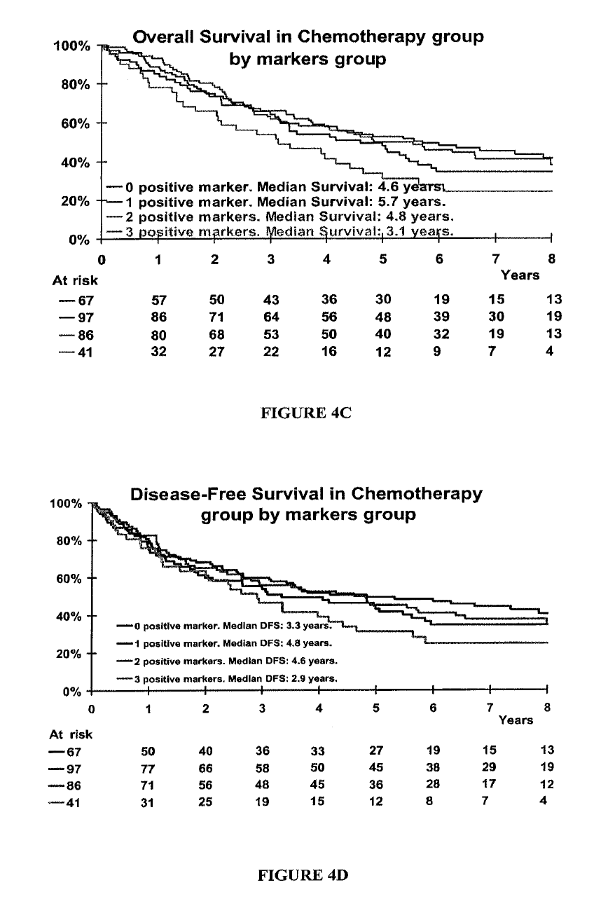

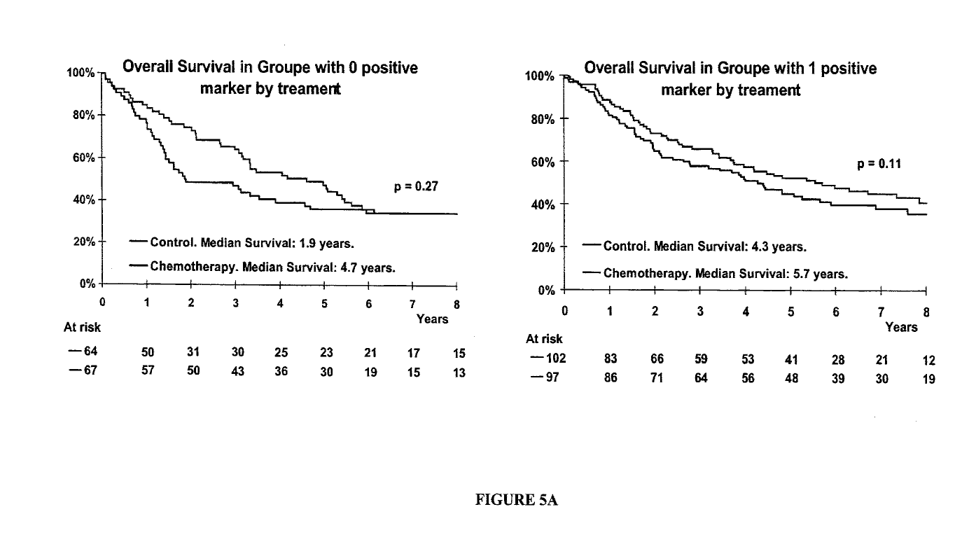

In the control arm, the overall survival of the 3 positive markers group compared to the 0 positive marker group was associated with an adjusted hazard ratio for death of 0.44 (95% CI, 0.24 to 0.80; P=0.01). Median overall survival was 69 months (5.8 years) longer in the 3 positive markers group (92 months, 7.7 years) than in the 0 positive marker group (23 months, 1.9 years) (FIG. 4A).

The disease-free survival of the 3 positive markers group compared to the 0 positive marker group was associated with an adjusted hazard ratio for death of 0.36 (95% CI, 0.20 to 0.65; P=0.0007). Median disease-free survival was 77 months (6.4 years) longer in the 3 positive markers group (94 months, 7.8 years) than in the 0 positive marker group (17 months, 1.4 years) (FIG. 4B).

In the chemotherapy arm, there was no difference in overall survival between the 3 positive markers group and the 0 positive marker group (adjusted hazard ratio for death, 1.38; 95% CI, 0.83 to 2.30; P=0.21) (FIG. 4C). Indeed, there was no difference in disease-free survival between the 3 positive markers group and the 0 positive marker group (adjusted hazard ratio for death, 1.20; 95% CI, 0.73 to 1.99; P=0.47) (FIG. 4D).

The results obtained in the control arm (patients that had received no chemotherapy) demonstrate that 1) the patients with 3 positive markers (PARP-positive/MSH2-positive/ERCC1-positive) had an excellent long-term survival; 2) bring to light a particular subgroup, the 0 positive marker group (PARP-negative/MSH2-negative/ERCC1-negative), that had a very short survival and need to be followed with attention.

The results obtained demonstrate that PARP/MSH2/ERCC1 combination is a strong powerful prognostic signature in patients under observation.

### C) Effect of Chemotherapy on Overall and Disease-Free Survival According to PARP, MSH2 and ERCC1 Expression and Predictive Value of Combining PARP, MSH2 and ERCC1

The expression levels of PARP, MSH2 and ERCC1 were together available for 570 patients. In a sub-analysis of these particular patients, the effect of chemotherapy was examined in the following subgroups defined by combining PARP, MSH2 and ERCC1 expression:
0 positive marker (PARP-negative/MSH2-negative/ERCC1-negative)
1 positive marker
2 positive markers and
3 positive markers (PARP-positive/MSH2-positive/ERCC1-positive).

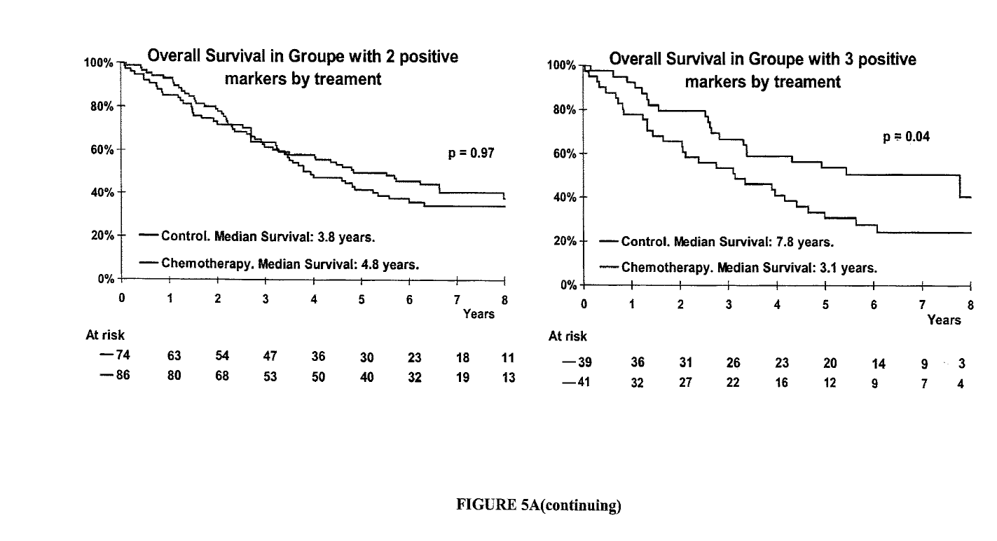

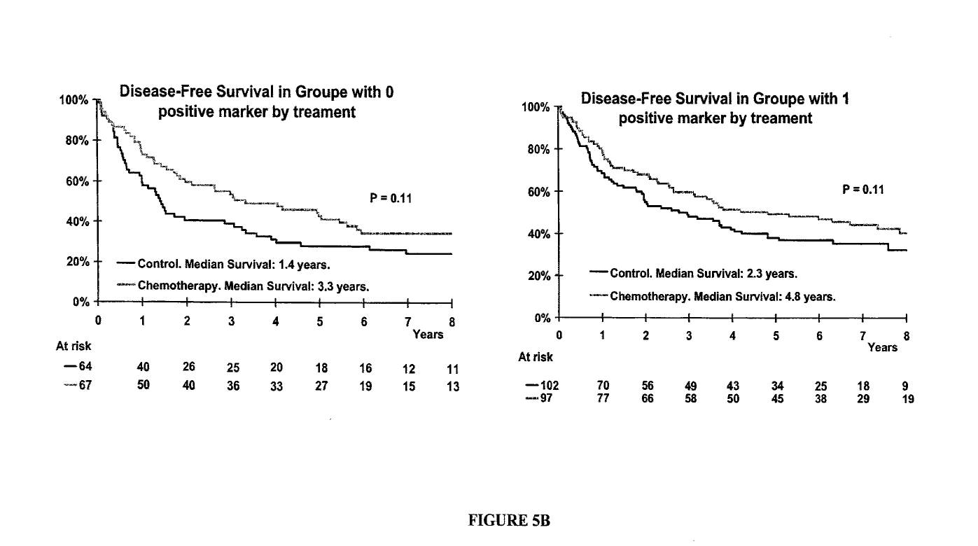

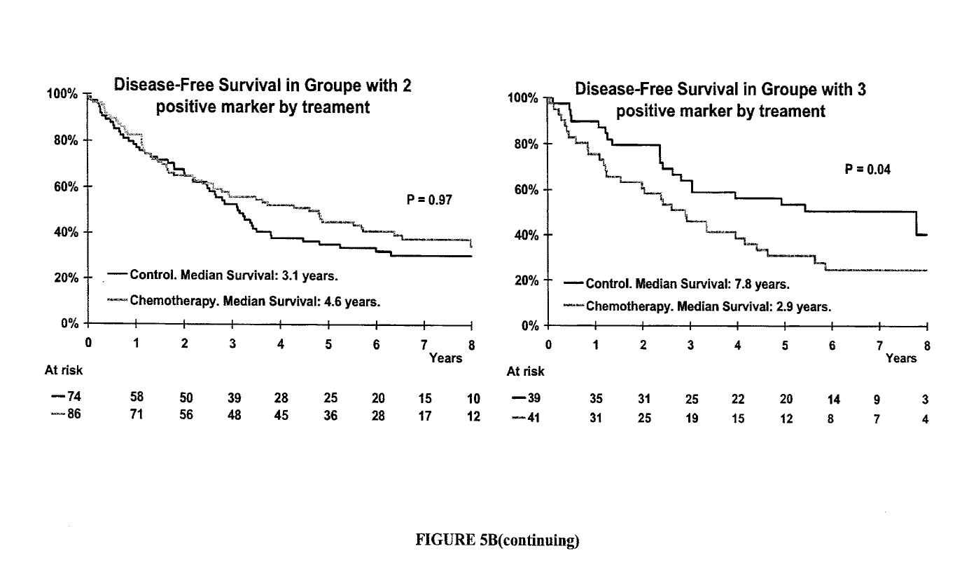

In the 3 positive markers group (80 patients, 14% of the patients included in IALT-Bio) for the overall survival, the adjusted hazard ratio for death associated with chemotherapy versus observation was 1.84 (95% CI, 1.04 to 3.25; P=0.04) (Table 4). In the 3 positive markers group, median overall survival was 56 months (4.7 years) longer in the control arm (93 months, 7.8 years) than in the chemotherapy group (37 months, 3.1 years) (FIG. 5A).

TABLE 4
Overall survival according to allocated
treatment and subgroup analysis

| | CT group | Control group | HR for deaths |
|---|---|---|---|
| | N = 291 | N = 279 | CT vs. no CT |
| | No Deaths/ | No Deaths/ | [95% CI] |
| Overall Survival | No patients | No patients | p-value |
| Group 0 | 43/67 | 45/64 | 0.78 |
| N = 131 | | | [0.51; 1.21] |
| | | | 0.27 |
| Group 1 | 54/97 | 63/102 | 0.74 |
| N = 199 | | | [0.51; 1.07] |
| | | | 0.11 |
| Group 2 | 51/86 | 49/74 | 0.99 |
| N = 160 | | | [0.67; 1.48] |
| | | | 0.97 |
| Group 3 | 31/41 | 20/39 | 1.84 |
| N = 80 | | | [1.04; 3.25] |
| | | | 0.04 |

Heterogeneity Test: p = 0.05
Trend Test: p = 0.02

In the 3 positive markers group (80 patients, 14% of the patients included in IALT-Bio) for the disease-free survival, the adjusted hazard ratio for death associated with chemotherapy versus observation was 1.82 (95% CI, 1.03 to 3.23; P=0.04) (Table 5). In the 3 positive markers group, median disease-free survival was 59 months (4.9 years) longer in the control arm (94 months, 7.8 years) than in the chemotherapy group (35 months, 2.9 years) (FIG. 5B).

TABLE 5
Disease-free survival according to allocated
treatment and subgroup analysis

| | CT group | Control group | HR for events |
|---|---|---|---|
| | N = 291 | N = 279 | CT vs. noCT |
| Disease-Free | No events/ | No events/ | [95% CI] |
| Survival | No patients | No patients | p-value |
| Group 0 | 43/67 | 49/64 | 0.71 |
| N = 131 | | | [0.46; 1.08] |
| | | | p = 0.11 |
| Group 1 | 55/97 | 66/102 | 0.74 |
| N = 199 | | | [0.52; 1.07] |
| | | | p = 0.11 |
| Group 2 | 55/86 | 52/74 | 0.99 |
| N = 160 | | | [0.68; 1.46] |
| | | | p = 0.97 |
| Group 3 | 31/41 | 20/39 | 1.82 |
| N = 80 | | | [1.03; 3.23] |
| | | | p = 0.04 |

Heterogeneity Test: p = 0.04.
Trend Test: p < 0.01.

These results demonstrated that the chemotherapy have a fatal effect in the 3 positive markers group (PARP-positive/MSH2-positive/ERCC1-positive group), the median survival is decreased to 4.9 years when this group of patients received platinum-based adjuvant chemotherapy. The results demonstrate that PARP/MSH2/ERCC1 signature is a predictor of overall and disease-free survival benefit from cisplatin-based chemotherapy.

The results obtained demonstrate that the long-term survival benefit derived from adjuvant chemotherapy may be different according to PARP expression in cancer patients, particularly in NSCLC. Patients in the PARP-positive group did not benefit from chemotherapy, while patients in the PARP-negative group did. In the PARP-negative group, there was a gain in median survival in favor of chemotherapy versus observation. The results obtained demonstrate that PARP is prognostic in patients under observation and is a predictor of overall survival benefit from cisplatin-based chemotherapy.

These results also demonstrate that the long-term survival benefit derived from adjuvant chemotherapy may be different according to MSH2 expression in cancer patients, particularly in NSCLC. Patients in the MSH2-positive group did not benefit from chemotherapy, while patients in the MSH2-negative group did. In the MSH2-negative group, there was a reduced adjusted hazard ratio for death of 25%, and a gain in median survival of 16 months in favor of chemotherapy versus observation. When the analysis focused exclusively on the control arm, MSH2 positivity was associated with longer overall survival. The results are strengthened by the adjustments for multivariate overall survival predictors and by the use of two-sided statistical tests. They demonstrate also that MSH2 is prognostic in patients under observation and is a predictor of overall survival benefit from cisplatin-based chemotherapy.

A previous study already demonstrated a survival benefit from adjuvant cisplatin-based chemotherapy in patients whose tumors were ERCC1 negative which was not the case for patients with ERCC1-positive lesions¹⁸. Furthermore, the prognostic role of ERCC1 tumor expression suggested by our data was later strongly supported by Zheng et al²⁰. In the present study, we used recently updated survival data from the IALT study (7.5 years of median survival) which for the first time allowed us to evaluate DNA repair markers as predictors of the long-term benefit of cisplatin-based chemotherapy¹⁹.

PARP, MSH2 and ERCC1 are involved in the repair of cisplatin-induced DNA lesions^{5, 6, 27}. Here, the inventors have demonstrated that they are coordinately expressed. The differential effect of chemotherapy on patients with PARP-positive and PARP-negative tumors, and with MSH2-positive and MSH2-negative tumors was similar among patients with ERCC1-positive and ERCC1-negative tumors, suggesting that the predictive value of PARP is partly independent of that of MSH2 and/or ERCC1. Indeed, when PARP marker and at least one of the MSH2 or ERCC1 markers were negative, the long-term effect of chemotherapy was associated with a reduced adjusted hazard ratio for death which highly suggests that the predictive value of PARP and that of MSH2 and/or ERCC1 act cumulatively.

Moreover, when PARP, MSH2 and ERCC1 markers were positive, the long-term effect of chemotherapy was associated with an increased adjusted hazard ratio for death which highly suggests that the predictive value of PARP and MSH2 and ERCC1 act cumulatively.

In conclusion, the results of the present study indicate that PARP, preferably together with MSH2, more preferably together with MSH2 and ERCC1, is prognostic in patients under observation and predictive of long-term overall and disease-free survival benefit from platinum-based chemotherapy, particularly for cisplatin-based chemotherapy. These results also demonstrate the potential use of PARP, preferably together with MSH2, more preferably together with MSH2 and ERCC1 in the clinical setting.

### REFERENCES

1. Gazdar A F. DNA repair and survival in lung cancer—the two faces of Janus. The New England journal of medicine 2007; 356(8):771-3.
2. Fishel R, Lescoe M K, Rao M R, et al. The human mutator gene homolog MSH2 and its association with hereditary nonpolyposis colon cancer. Cell 1993; 75(5):1027-38.
3. Duckett D R, Drummond J T, Murchie A I, et al. Human MutSalpha recognizes damaged DNA base pairs containing O6-methylguanine, O4-methylthymine, or the cisplatin-d(GpG) adduct. Proc Natl Acad Sci USA 1996; 93(13):6443-7.
4. Zdraveski Z Z, Mello J A, Farinelli C K, Essigmann J M, Marinus M G. MutS preferentially recognizes cisplatin-over oxaliplatin-modified DNA. J Biol Chem 2002; 277(2): 1255-60.
5. Lan L, Hayashi T, Rabeya R M, et al. Functional and physical interactions between ERCC1 and MSH2 complexes for resistance to cis-diamminedichloroplatinum(II) in mammalian cells. DNA Repair (Amst) 2004; 3(2):135-43.
6. Zhang N, Liu X, Li L, Legerski R. Double-strand breaks induce homologous recombinational repair of interstrand cross-links via cooperation of MSH2, ERCC1-XPF, REV3, and the Fanconi anemia pathway. DNA Repair (Amst) 2007; 6(11):1670-8.
7. Xinarianos G, Liloglou T, Prime W, et al. hMLH1 and hMSH2 expression correlates with allelic imbalance on chromosome 3p in non-small cell lung carcinomas. Cancer Res 2000; 60(15):4216-21.
8. Brooks K R, To K, Joshi M B, et al. Measurement of chemoresistance markers in patients with stage III non-small cell lung cancer: a novel approach for patient selection. Ann Thorac Surg 2003; 76(1):187-93; discussion 93.
9. Wang Y C, Lu Y P, Tseng R C, et al. Inactivation of hMLH1 and hMSH2 by promoter methylation in primary non-small cell lung tumors and matched sputum samples. J Clin Invest 2003; 111(6):887-95.
10. Zhu L Q, Diao L M, Chen D J, Li H G, Liu X, Zou Z Y, Li B Y, Wang M, Liu M Q. Relationship between hMSH2 and FHIT gene expression in non-small cell lung cancer Ai Zheng. 2003; 22(6):571-4.
11. Hsu H S, Wen C K, Tang Y A, et al. Promoter hypermethylation is the predominant mechanism in hMLH1 and hMSH2 deregulation and is a poor prognostic factor in nonsmoking lung cancer. Clin Cancer Res 2005; 11(15):5410-6.
12. Skarda J, Fridman E, Plevova P, et al. Prognostic value of hMLH1 and hMSH2 immunohistochemical expression in non-small cell lung cancer. A tissue microarray study. Biomed Pap Med Fac Univ Palacky Olomouc Czech Repub 2006; 150(2):255-9.
13. Kanellis G, Chatzistamou I, Koutselini H, et al. Expression of DNA mismatch repair gene MSH2 in cytological material from lung cancer patients. Diagn Cytopathol 2006; 34(7):463-6.
14. Scartozzi M, Franciosi V, Campanini N, et al. Mismatch repair system (MMR) status correlates with response and survival in non-small cell lung cancer (NSCLC) patients. Lung Cancer 2006; 53(1):103-9.
15. Hsu H S, Lee I H, Hsu W H, Kao W T, Wang Y C. Polymorphism in the hMSH2 gene (gISV12-6T>C) is a prognostic factor in non-small cell lung cancer. Lung Cancer 2007; 58(1):123-30.
16. Cooper W A, Kohonen-Corish M R, Chan C, et al. Prognostic significance of DNA repair proteins MLH1, MSH2 and MGMT expression in non-small-cell lung cancer and precursor lesions. Histopathology 2008; 52(5):613-22.
17. Arriagada R, Bergman B, Dunant A, Le Chevalier T, Pignon J P, Vansteenkiste J. Cisplatin-based adjuvant chemotherapy in patients with completely resected non-small-cell lung cancer. The New England journal of medicine 2004; 350(4):351-60.
18. Olaussen K A, Dunant A, Fouret P, et al. DNA repair by ERCC1 in non-small-cell lung cancer and cisplatin-based adjuvant chemotherapy. The New England journal of medicine 2006; 355(10):983-91.
19. Le Chevalier T, Dunant A, Arriagada R, et al. Long-term results of the International Adjuvant Lung Cancer Trial (IALT) evaluating adjuvant cisplatin-based chemotherapy in non-small cell lung cancer (NSCLC). J Clin Oncol 2008; 26(15 S Suppl): 398s.

20. Zheng Z, Chen T, Li X, Haura E, Sharma A, Bepler G. DNA synthesis and repair genes RRM1 and ERCC1 in lung cancer. The New England journal of medicine 2007; 356(8):800-8.
21. Forgacs E, Wren J D, Kamibayashi C, et al. Searching for microsatellite mutations in coding regions in lung, breast, ovarian and colorectal cancers. Oncogene 2001; 20(8):1005-9.
22. Weinberg R A. The biology of cancer. Garland Science Eds 2007:463-556.
23. Leppard J. B., Dong Z, Mackey Z. B., Tomkinson A. E., Physical and functional interaction between DNA ligase IIIalpha and poly(ADP-Ribose) polymerase 1 in DNA single-strand break repair, Mol. Cell. Biol. 23 (2003) 5919-5927.
24. Sugimura K, Takebayashi S, Taguchi H, Takeda S, Okumura K. PARP-1 ensures regulation of replication fork progression by homologous recombination on damaged DNA. J. Cell Biol. 2008 Dec. 29; 183(7):1203-12.
25. de Murcia, J. M., C. Niedergang, C. Trucco, M. Ricoul, B. Dutrillaux, M. Mark, F. J. Oliver, M. Masson, A. Dierich, M. LeMeur, et al. 1997. Requirement of poly(ADP-ribose) polymerase in recovery from DNA damage in mice and in cells. Proc. Natl. Acad. Sci. USA. 94:7303-7307.
26. de Murcia, J., M. Ricoul, L. Tartier, C. Niedergang, A. Huber, F. Dantzer, V. Schreiber, J.C. Ame, A. Dierich, M. LeMeur, et al. 2003. Functional interaction between PARP-1 and PARP-2 in chromosome stability and embryonic development in mouse. EMBO J. 22:2255-2263.
27. Guggenheim E R, Xu D, Zhang C X, Chang P V, Lippard S J. Photoaffinity isolation and identification of proteins in cancer cell extracts that bind to platinum-modified DNA. Chembiochem. 2009 Jan. 5; 10(1):141-57.
28. Miwa M, Masutani M. PolyADP-ribosylation and cancer. Cancer Sci. 2007 October; 98(10):1528-35.
29. Kamal N S, Soria J C, Mendiboure J, Planchard D, Olaussen K A, Rousseau V, Popper H, Piker R, Bertrand P, Dunant A, Le Chevalier T, Filipits M, Fouret P. MutS Homologue 2 and the Long-term Benefit of Adjuvant Chemotherapy in Lung Cancer. Clin Cancer Res; 16(4); 1206-15.

1. An in vitro method for predicting the benefit of the response of a subject diagnosed with cancer to a platinum-based chemotherapy comprising:
a) obtaining a biological sample from a subject;
b) determining the poly (ADP-ribose) polymerase (PARP) expression level in the biological sample; and
c) comparing said PARP expression level to the PARP expression level of a reference control or population, or comparing to a non tumor cell control.
wherein a PARP expression level that is less than or equal to the expression level obtained for said reference control or population, or is decreased compared to the non tumor cell content, predicts an overall survival and/or a disease-free survival benefit;
and wherein a PARP expression level that is greater than the expression level obtained for said reference control or population, or is increased compared to the non tumor cell content, does not predict an overall survival and/or a disease-free survival benefit.

2. The method according to claim 1, further comprising:
a) determining the MSH2 and/or ERCC1 expression level from the same or from another biological sample from the subject; and
b) optionally, comparing said MSH2 and/or ERCC1 expression level to the MSH2 and/or ERCC1 expression level of a reference control or population, or compared to a non tumor cell control cells content;
wherein a MSH2 and/or ERCC1 expression level that is less than or equal to the expression level obtained for said reference population, or is decreased compared to the non tumor cell control, predicts an overall survival and/or a disease-free survival benefit;
and wherein a MSH2 and/or ERCC1 expression level that is greater than the expression level obtained for said reference population, or is increased compared to the non tumor cell control, does not predict an overall survival and/or a disease-free survival benefit.

3.-6. (canceled)

7. An in vitro method for assessing whether a platinum-based chemotherapy is appropriate for a subject diagnosed with cancer comprising:
a) obtaining a biological sample from a subject;
b), determining the PARP, and optionally the MSH2 and/or ERCC1, expression level in said biological sample; and
c) optionally, comparing said PARP expression level, and optionally the MSH2 and/or ERCC1 expression level, to a reference control or population, or to a non-tumor cell control;
wherein a platinum-based chemotherapy will be determined to be an appropriate chemotherapy if the PARP expression level, and optionally the MSH2 and/or ERCC1 expression level, is less than or equal to the PARP expression level, and optionally the MSH2 and/or ERCC1 expression level, of a reference control or population, or is decreased compared to the non tumor cell control,
and wherein a platinum-free chemotherapy will be determined to be an appropriate chemotherapy if the PARP expression level, and optionally the MSH2 and/or ERCC1 expression level, is greater than the PARP expression level and optionally the MSH2 and/or ERCC1 expression level, of said reference control or population, or is increased compared to the non tumor cell control.

8.-12. (canceled)

13. The method as in claim 1 or 7 wherein said subject is a human.

14. The method as in claim 1 or 7, wherein said chemotherapy is an adjuvant-platinum-based chemotherapy.

15. The method as in claim 1 or 7, wherein said platinum-based chemotherapy is cisplatin-based chemotherapy.

16.-17. (canceled)

18. The method as in claim 1 or 7, wherein the subject has a malignant mesothelioma, a bladder cancer, a testicular cancer, cancer of the upper aero-digestive tract, non-small-cell lung cancer (NSCLC) or ovarian cancer.

19. (canceled)

20. The method as in claim 1 or 7, wherein said biological sample is a tissue sample comprising cancer cells, or a biopsy containing tumor cells.

21. The method of claim 7, wherein said PARP, MSH2 or ERCC1 expression level is determined by measuring PARP, MSH2 or ERCC1 RNA transcripts or PARP, MSH2 or ERCC1 protein, respectively.

22. The method of claim 21 wherein the PARP, MSH2 or ERCC1 expression level is determined by PCR, RT-PCR, Northern, Western, or immunohistochemistry.

23. A kit or array comprising:
a reagent for assaying PARP expression; and
a reagent for assaying MSH2 and/or a reagent for assaying ERCC1 expression.

24. (canceled)

25. The kit or array of claim 23, comprising a detection agent selected from the group consisting of:
a) a probe and/or or a pair of primers that specifically hybridizes to the PARP mRNA or cDNA, or to the complementary sequence thereof, and
b) a probe and/or or a pair of primers that specifically hybridizes to the MSH2 mRNA or cDNA, or to the complementary sequence thereof,
c) a probe and/or or a pair of primers that specifically hybridizes to the ERCC1 mRNA or cDNA, or to the complementary sequence thereof,
d) an anti-PARP antibody, optionally labeled, capable of specifically recognizing the PARP protein,
e) an anti-MSH2 antibody, optionally labeled, capable of specifically recognizing the MSH2 protein, and
f) an anti-ERCC1 antibody, optionally labeled, capable of specifically recognizing the ERCC1 protein.

26.-28. (canceled)

29. The method as in claim 1 or 7, wherein said PARP is the poly (ADP-ribose) polymerase 1 (PARP1).

30. The kit or array of claim 23, wherein said PARP is the poly (ADP-ribose) polymerase 1 (PARP1).
